(19) United States
(12) Patent Application Publication
(10) Pub. No.: US 2018/0092864 A1
(43) Pub. Date: Apr. 5, 2018

(54) COMPOSITIONS AND METHODS FOR TREATING SEIZURE DISORDERS

(71) Applicant: ZOGENIX INTERNATIONAL LIMITED, Berkshire (GB)

(72) Inventors: Parthena MARTIN, Emeryville, CA (US); Brooks M. BOYD, Berkeley, CA (US); Arnold GAMMAITONI, Emeryville, CA (US); Bradley S. Galer, West Chester, PA (US); Gail FARFEL, Emeryville, CA (US)

(73) Assignee: ZOGENIX INTERNATIONAL LIMITED, Berkshire (GB)

(21) Appl. No.: 15/717,159

(22) Filed: Sep. 27, 2017

Related U.S. Application Data
(60) Provisional application No. 62/402,881, filed on Sep. 30, 2016.

(51) Int. Cl. A61K 31/137; A61K 9/00

(52) U.S. Cl. CPC A61K 31/137; A61K 9/0053

(57) ABSTRACT

Functional analogs of fenfluramine are provided. The subject fenfluramine functional analogs find use in the treatment of a variety of diseases. For example, methods of treating epilepsy by administering a fenfluramine analog to a subject in need thereof are provided. Also provided are methods of treating a neurodegenerative disease in a subject in need thereof. Pharmaceutical compositions for use in practicing the subject methods are also provided.

### FIELD OF THE INVENTION

The present invention relates generally to the therapeutic treatment of patients diagnosed with a seizure disorder. More specifically, the invention relates to therapeutic agents that are functional analogs of the amphetamine drug fenfluramine, and to methods of using those compounds to treat human patients diagnosed with intractable forms of epilepsy.

### BACKGROUND OF THE INVENTION

Epilepsy is a condition of the brain marked by a susceptibility to recurrent seizures. There are numerous causes of epilepsy including, but not limited to birth trauma, perinatal infection, anoxia, infectious diseases, ingestion of toxins, tumors of the brain, inherited disorders or degenerative disease, head injury or trauma, metabolic disorders, cerebrovascular accident and alcohol withdrawal.

A large number of subtypes of epilepsy have been characterized, each with its own unique clinical symptoms, signs, and phenotype, underlying pathophysiology and distinct responses to different treatments. The most recent version, and the one that is widely accepted in the art, is the system adopted by the International League Against Epilepsy's (“ILAE”) Commission on Classification and Terminology (See e.g., Berg et. al, “Revised terminology and concepts for organization of seizures,” Epilepsia, 51(4):676-685 (2010)):

TABLE 1
ILAE Classification Scheme for Epilepsy Subtypes
I. ELECTROCHEMICAL SYNDROMES (by age of onset)

| | |
|---|---|
| A. Neonatal period | 1. Benign familial neonatal epilepsy (BFNE) |
| | 2. Early myoclonic encephalopathy (EME) |
| | 3. Ohtahara syndrome |
| B. Infancy | 1. Epilepsy of infancy with migrating focal |
| | seizures |
| | 2. West syndrome |
| | 3. Myoclonic epilepsy in infancy (MEI) |
| | 4. Benign infantile epilepsy |
| | 5. Benign familial infantile epilepsy |
| | 6. Dravet syndrome |
| | 7. Myoclonic encephalopathy in non- |
| | progressive disorders |
| C. Childhood | 1. Febrile seizures plus (FS+) (can start in |
| | infancy) |
| | 2. Panayiotopoulos syndrome |
| | 3. Epilepsy with myoclonic atonic (previously |
| | astatic) seizures (Doose syndrome) |
| | 4. Benign epilepsy with centrotemporal |
| | spikes (BECTS) |
| | 5. Autosomal-dominant nocturnal frontal |
| | lobe epilepsy (ADNFLE) |
| | 6. Late onset childhood occipital epilepsy |
| | (Gastaut type) |
| | 7. Epilepsy with myoclonic absences |
| | 8. Lennox-Gastaut syndrome |
| | 9. Epileptic encephalopathy with continuous |
| | spike-and-wave during |
| | 10. Landau-Kleffner syndrome (LKS) |
| | 11. Childhood absence epilepsy (CAE) |
| D. Adolescence- | 1. Familial focal epilepsy with variable foci |
| Adult | (childhood to adult) |
| | 2. Reflex epilepsies |
| E. Less specific | 1. Familial focal epilepsy with variable foci |
| age relationship | (childhood to adult) |
| | 2. Reflex epilepsies |

II. DISTINCTIVE CONSTELLATIONS

| | |
|---|---|
| A. Mesial temporal | |
| lobe epilepsy with | |
| hippocampal sclerosis | |
| (MTLE with HS) | |
| B. Rasmussen | |
| syndrome | |
| C. Gelastic seizures | |
| with hypothalamic | |
| hamartoma | |
| D. Hemiconvulsion- | |
| hemiplegia-epilepsy | |
| E. Epilepsies that | 1. Presumed cause (presence or absence of |
| do not fit into any | a known structural or metabolic condition) |
| of these diagnostic | 2. Primary mode of seizure onset (generalized |
| categories, | vs. focal) |

III. EPILEPSIES ATTRIBUTED TO AND ORGANIZED
BY STRUCTURAL-METABOLIC CAUSES

A. Malformations of
cortical development
(hemimegalencephaly,
heterotopias, etc.,)
B. Neurocutaneous
syndromes (tuberous
sclerosis complex,
Sturge-Weber, etc.,)
C. Tumor
D. Infection
E. Trauma

IV. ANGIOMA

A. Perinatal insults
B. Stroke
C. Other causes

V. EPILEPSIES OF UNKNOWN CAUSE
VI. CONDITIONS WITH EPILEPTIC SEIZURES NOT
TRADITIONALLY DIAGNOSED AS FORMS OF EPILEPSY PER SE

A. Benign neonatal
seizures (BNS)
B. Febrile seizures (FS)

Part V of the ILAE classification scheme underscores the fact that the list is far from complete, and that there are still subtypes of epilepsy that have not yet been fully characterized, or that remain unrecognized as distinct syndromes. That is to say, those skilled in the art will recognize that different subtypes of epilepsy are triggered by different stimuli, are controlled by different biological pathways, and have different causes, whether genetic, environmental, and/or due to disease or injury of the brain. In other words, the skilled artisan will recognize that teachings relating to one epileptic subtype are most commonly not necessarily applicable to any other subtype.

Of particular importance is that there are a large number of compounds that are used to treat different types of epilepsy, and that different epilepsy subtypes respond differently to different anticonvulsant drugs. That is, while a particular drug can be effective against one form of epilepsy, it can be wholly ineffective against others, or even contra-indicated due to exacerbation of symptoms, such as worsening the frequency and severity of the seizures. As a result, efficacy of a particular drug with respect to a particular type of epilepsy is wholly unpredictable, and therefore it is an entirely surprising discovery when a particular drug not previously known to be effective for a particular type of epilepsy is found to be effective. This is especially true for those epilepsy syndromes which were previously intractable and resistant to known drugs.

There are a large number of different drugs which have been used in the treatment of various forms of epilepsy. Although the list below is not comprehensive, it is believed to include those drugs which are widely prescribed in patients diagnosed with epilepsy.

TABLE 2
Commonly Prescribed Antiepileptic Drugs

| Generic Name | Trade Name |
|---|---|
| carbamazepine | Carbatrol, Epitol, Equetro, Tegretol |
| clobazam | Frisium, Onfi |
| clonazepam | Klonopin |
| diazepam | Diastat, Valium |
| ezogabine | Potiga |
| eslicarbazepine | Aptiom |
| acetate | |
| ethosuximide | Zarontin |
| felbamate | Felbatol |
| fosphenytoin | Cerebyx |
| gabapentin | Gralise, Horizant, Neurontin, Gabarone |
| Lacosamide | Vimpat |
| lamotrigine | LaMICtal |
| levetiracetam | Elepsia, Keppra, Levetiractam Stavzor |
| lorazepam | Ativan |
| oxcarbazepine | Trileptal, Oxtellar |
| perampanel | Fycompa |
| phenobarbital | Luminal, Solfoton |
| phenytoin | Dilations, Prompt, Di-Phen, Epanutin, Phenytek |
| pregabalin | Lyrica |
| primidone | Mysoline |
| rufinamide | Banzel, Inovelon |
| tiagabine | Gabitril |
| topiramate | Qudexy XR, Topamax, Topiragen, Trokendi XR, |
| valproate, | Depacon, Depakene, Depakote, |
| valproic acid | |
| vigabatrin | Sabril |
| zonisamide | Zonegran |

Thus, there is a large number of drugs of diverse types that which have been used in the treatment of various forms of epilepsy, and different epilepsy subtypes respond differently to different anticonvulsant drugs. Thus, persons of ordinary skill in the art recognize that whether a patient with a particular type of epilepsy will respond to a particular drug is not predictable, and hence the efficacy of a particular drug for a particularly type of epilepsy is in all cases a surprising result.

Dravet Syndrome is a rare and catastrophic form of intractable epilepsy that begins in infancy. Initially, the patient experiences prolonged seizures. In their second year, additional types of seizure begin to occur and this typically coincides with a developmental decline or stagnation, possibly due to repeated cerebral hypoxia resulting from ongoing relentless seizures. This leads to poor development of language and motor skills.

Children with Dravet Syndrome are likely to experience multiple seizures per day. Epileptic seizures are far more likely to result in death in sufferers of Dravet Syndrome; approximately 10 to 15% of patients diagnosed with Dravet Syndrome die in childhood, particularly between two and four years of age. Additionally, patients are at risk of numerous associated conditions including orthopedic developmental issues, impaired growth and chronic upper respiratory infections.

The cost of care for Dravet Syndrome patients is also high as the affected children require constant supervision and many require institutionalization as they reach teenage years.

The presentation and diagnosis of Dravet syndrome differs significantly from other forms of epilepsy. Ceulemans teaches that Dravet syndrome can be distinguished from other forms of epilepsy by:

“ . . . the appearance of tonic-clonic seizures during the first year of life, the occurrence of myoclonic seizures and ataxia later, impaired psychomotor development following the onset of the seizures, and poor response to anti-epileptic drugs.” Ceulemans, Developmental Medicine & Child Neurology, 2011, 53, 19-23, PTO-892.

Brunklaus et. al, (BRAIN, 2012, pages 1-8, PTO-892) similarly observes: “Dravet syndrome typically presents in the first year of life with prolonged, febrile and afebrile, generalized clonic or hemiclonic epileptic seizures in children with no pre-existing developmental problems. Other seizure types including myoclonic, focal and atypical absence seizures appear between the ages of 1 and 4 years (Dravet, 1978).”

Thus, the presentation and diagnosis of Dravet syndrome is significantly different from other forms of epilepsy. Given its distinctive clinical nature, one of ordinary skill in the art would therefore not find it obvious or have reason to assume that any particular compound would be efficacious in Dravet syndrome.

Dravet is also distinctive in terms of its genetic aspects. It is known in the art (Ceulemans, Developmental Medicine & Child Neurology, 2011, 53, 19-23, PTO-892, Brunklaus et al. (BRAIN, 2012, pages 1-8, PTO-892) that mutations in the alpha-subunit of the neuron-specific voltage-gated sodium channel (SCN1a) was discovered as the primary genetic cause for Dravet syndrome in 2001. Thus, the cause of Dravet syndrome is significantly different as compared to other forms of epilepsy. Moreover, unlike other forms of epilepsy, diagnosis of Dravet is based in part on detection of these genetic mutations in addition to clinical observation. Consequently, with the advent of improved genetic testing, there has been an increase in the number of patients diagnosed with the disease.

Of particular concern, children with Dravet Syndrome are particularly susceptible to episodes of Status Epilepticus. This severe and intractable condition is categorized as a medical emergency requiring immediate medical intervention, typically involving hospitalization. Status Epilepticus can be fatal. It can also be associated with cerebral hypoxia, possibly leading to damage to brain tissue. Frequent hospitalizations of children with Dravet Syndrome are clearly distressing, not only to the patient but also to family and care givers.

Although a number of anticonvulsant therapies have been employed to reduce the instance of seizures in patients with Dravet Syndrome, the results obtained with such therapies are typically poor and those therapies only affect partial cessation of seizures at best. In general, seizures associated with Dravet Syndrome are typically resistant to conventional treatments, and anticonvulsants whose activity is via blockade of the sodium channel worsen seizures in Dravet syndrome. Further, many anticonvulsants such as clobazam and clonazepam have undesirable side effects, which are particularly acute in pediatric patients.

It has recently been discovered that the intractable seizures characteristic of Dravet syndrome can be significantly reduced in frequency and/or severity, and in some cases eliminated entirely, by administering the drug 3-trifluoromethyl-N-ethylamphetamine (hereinafter “fenfluramine”). See Ceulemans et. al., Successful use of fenfluramine as an add-on treatment for Dravet Syndrome, Epilepsia 53(7):1131-1139, 2012. Fenfluramine, is an amphetamine derivative having the following structure:

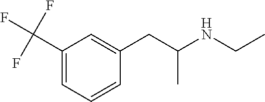 [SMILES: CCNC(C)Cc1cccc(C(F)(F)F)c1]

### Structure 1

### (RS)—N-ethyl-1-[3-(trifluoromethyl)phenyl]propan-2-amine

Fenfluramine was known to have high affinity for and activity at the 5-HT2A, 5-HT2B and 5-HT2C receptor subtypes (Rothman et al, 2015). 5-HT2C-agonists trigger appetite suppression, and therefore fenfluramine was used for treating obesity by co-administering it together with phentermine as part of the popular weight loss drug combination treatment marketed as Fen-Phen (i.e., fenfluramine/phentermine). Subsequently, Fen-Phen was withdrawn from sale globally and is not currently indicated for use in any therapeutic area.

Both fenfluramine and, more potently, fenfluramine's primary metabolite norfenfluramine, also activate the 5-HT2B receptor, Activation of the 5-HT2B receptor has been associated with cardiac valve hypertrophy. It was this drug-induced valvulopathy that resulted in the withdrawal of fenfluramine from the market in September of 1997. Hence, while fenfluramine is effective as an anti-seizure medication, it also has the potential for causing serious side effects. Patients who receive fenfluramine must be carefully monitored, which is time-consuming and expensive. Further, fenfluramine is contra-indicated for patients who are at higher risk of developing valvulopathies, pulmonary hypertension, or are predisposed to other serious adverse effects; and the drug can be discontinued where the patient experiences those effects.

Thus, there is a dire, long felt, but previously unmet need for therapeutic agents effective in treating, preventing or ameliorating the frequent severe seizures suffered by patients with refractory epilepsy syndromes, including but not limited to Dravet syndrome, Lennox-Gastaut syndrome, and Doose syndrome, which are not associated with unwanted side effects. See Lagae et al., “Add-on Therapy with Low Dose Fanfluramine (ZX008) in Lennox Gastaut Syndrome” Abstract 3.366, 2016, presented at the AES 2016 Annual Meeting in Houston, Tex. (presenting the results of a single center Phase 2 pilot open label dose finding trial of fenfluramine as an add-on therapy; text and figures available at: https://www.aesnet.org/meetings_events/annual_meeting_abstracts/view/240065); see also U.S. patent application Ser. No. 15/246,346.

### BRIEF SUMMARY OF THE INVENTION

The compositions and methods provided herein meet that need. The present invention provides therapeutic agents that are functional analogs of fenfluramine (Appendix 1 that forms a part of this application) that act on multiple receptors and that are useful for treating, preventing or ameliorating symptoms associated with seizure disorders in a patient in need of such treatment. It further provides methods for practicing the disclosed methods, as well as pharmaceutical formulations and dosage forms comprising those agents. For example, the disclosed methods are useful in preventing, treating or ameliorating symptoms associated with refractory seizure disorders for which conventional antiepileptic drugs are inadequate, ineffective, or contraindicated, including but not limited to Dravet syndrome, Lennox-Gastaut syndrome, Doose syndrome.

The invention here is based on the surprising discovery that, in addition to having activity at several (5-HT) receptor sub-types, specifically the 5-HT1D, 5-HT2A, and 5-HT2C receptor sub-types, fenfluramine is also active at other receptors, in particular at the Sigma 1 receptor, the beta-2 adrenergic receptor, the Muscarinic M1 receptor and the voltage-gated Na channel protein Nav1.5. Based on their work in further elucidating the mechanism underlying fenfluramine's pharmaceutical effects, the inventors have identified compounds (Appendix 1 that forms a part of this application) active at one or more of those receptors as potential therapeutic candidates. Testing in animal models led to the unexpected discovery that certain of those candidates surprisingly reduced epileptiform activity in in vivo animal models.

Thus, the disclosure provides methods which employ certain therapeutic agents useful in treating patients diagnosed with a seizure disease or disorder who require treatment. The disclosure further provides methods which employ certain therapeutic agents useful in preventing, treating or ameliorating symptoms associated with seizure diseases or disorders in patients who require treatment.

The methods disclosed herein comprise administering a therapeutically effective amount of one or more therapeutic agents. A number of therapeutic agents can be employed in the methods of the present invention.

For example, in one aspect, the disclosure provides a method of treatment comprising administering a therapeutically effective amount of a therapeutic agent comprising a compound selected from Compounds 1-157, as shown in Appendix 1.

In one aspect, the disclosure provides a method of preventing, treating or ameliorating symptoms associated with seizure diseases or disorders in patient who require treatment, wherein the therapeutic agent is a compound that is active at one or more targets. In one aspect, the therapeutic agent comprises a compound that is active at one or more targets which are selected from the group consisting of (a) a 5-HT receptor protein selected from the group consisting of the 5-HT1A receptor, the 5-HT1D receptor, the 5-HT1E receptor, the 5-HT2A receptor, the 5-HT2C receptor, the 5-HT5A receptor, and the 5-HT7 receptor, (b) an adrenergic receptor protein selected from the beta-1 adrenergic receptor, and the beta-2 adrenergic receptor, (c) a muscarinic acetylcholine receptor protein selected from the group consisting of the M1 muscarinic acetylcholine receptor the M2 muscarinic acetylcholine receptor, the M3 muscarinic acetylcholine receptor, the M4 muscarinic acetylcholine receptor, and the M5 muscarinic acetylcholine receptor, (d) a chaperone protein selected from the group consisting of the sigma-1 receptor and the sigma-2 receptor, (e) a sodium channel subunit protein selected from the group consisting of the Nav 1.1 subunit, the Nav 1.2 subunit, the subunit, the Nav 1.3 subunit, the Nav 1.4 subunit, the Nav 1.5 subunit, the Nav 1.6 subunit, and the Nav 1.7 subunit, and (f) a neurotransmitter transport protein selected from the group consisting of a serotonin transporter (SET), a dopamine transporter (DAT), and a norepinephrine transporter (NET).

In one embodiment of this aspect, the therapeutic agent comprises a compound that is active at one or more the 5-HT1A receptor selected from the 5-HT1A receptor, the 5-HT1D receptor, the 5-HT2A receptor, and the 5-HT2C receptor.

In another embodiment of this aspect, the therapeutic agent is a chaperone protein that is active at the Sigma 1 receptor. In one aspect, the activity of the therapeutic agent is selected from the group consisting of positive allosteric modulation, allosteric agonism, positive ago-allosteric modulation, negative ago-allosteric modulation, and neutral ago-allosteric modulation. In one aspect, the therapeutic agent is a positive allosteric modulator of the sigma-1 receptor.

In another embodiment of this aspect, the therapeutic agent is active at the beta-2 adrenergic receptor. In one aspect, the therapeutic agent is active at the Muscarinic M1 receptor.

In another embodiment of this aspect, the therapeutic agent is active at one or more targets, or two or more targets, or three or more targets, or four or more targets, or five or more targets, or more.

For example, the therapeutic agent is active at one or more of the Sigma 1, the beta-2 adrenergic receptor, the Muscarinic M1 receptor, the 5-HT transporter (SERT), the norepinephrine transporter (NET), the dopaminergic transporter (DAT), and in addition is active at one or more 5-HT receptors selected from the group consisting of the 5-HT1A receptor, the 5-HT1D receptor, the 5-HT2A receptor, the 5-HT2C receptor, the 5-HT5 receptor, and the 5-HT7 receptor.

In a preferred embodiment, the therapeutic agent is active at the sigma-1 receptor and one or more one or more 5HT receptor selected from the group consisting of the 5-HT1A receptor, the 5-HT1D receptor, the 5-HT2A receptor and the 5-HT2C receptor, more preferably at a 5HT receptor selected from the group consisting of the 5-HT1A receptor, the 5-HT1D receptor, the 5-HT2A receptor, and the 5-HT2C receptor. In a particularly preferred embodiment, the therapeutic agent is active at all of the 5-HT2A receptor, the 5-HT2C receptor, and the Sigma 1 receptor.

In another embodiment of this aspect, the therapeutic target is a functional hybrid that is active at one or more neurotransmitter transport proteins selected from the group consisting of the 5-HT transporter (SERT), the norepinephrine transporter (NET), and the dopaminergic transporter (DAT).

In particular embodiments, the therapeutic agent is selected from the group consisting Compounds PAL 433, PAL 1122, PAL 1123, PAL 363, PAL 361, PAL 586, PAL 588, PAL 591, PAL 743, PAL 744, PAL 787, PAL 820, PAL 304, PAL 434, PAL 426, PAL 429, and PAL 550, as shown in the table appearing in FIG. 14A.

In particular embodiments of this aspect, the therapeutic agent is a compound according to the structure:

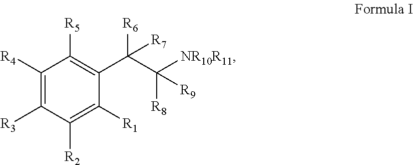

wherein

R1-R5 are each independently selected from H, OH, optionally substituted C1-4 alkyl, optionally substituted C1-3 alkoxy, optionally substituted C2-4 alkenyl, optionally substituted C2-4 alkynyl, halogen, amino, acylamido, CN, CF3, NO2, N3, CONH2, CO2R12, CH2OR12, NR12R13, NHCOR12, NHCO2R12, CONR12R13; C1-3 alkylthio, R12SO, R12SO2, CF3S, and CF3SO2;

R6 and R7 are each independently selected from H or optionally substituted C1-10alkyl, or R6 and R7 together constitute ═O or ═CH2;

R8 and R9 are each independently selected from H or optionally substituted C1-10alkyl;

R10, R11, R12, and R13 are each independently selected from H or optionally substituted C1-10 alkyl;

and wherein R1 and R8 may be joined to form a cyclic ring; or a pharmaceutically acceptable ester, amide, salt, solvate, prodrug, or isomer thereof,

with the proviso that when one of R8 and R9 is CH3, then at least one of R10 and R11 is optionally substituted C3-C10 cycloalkyl.

In particular embodiments of this aspect, the therapeutic agent is a compound according to the structure:

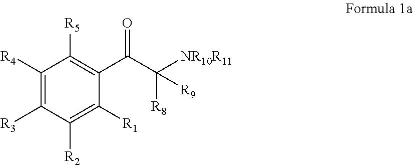

wherein

R1-R5 are each independently selected from H, OH, optionally substituted C1-4 alkyl, optionally substituted C1-3 alkoxy, optionally substituted C2-4 alkenyl, optionally substituted C2-4 alkynyl, halogen, amino, acylamido, CN, CF3, NO2, N3, CONH2, CO2R12, CH2OR12, NR12R13, NHCOR12, NHCO2R12, CONR12R13; C1-3 alkylthio, R12SO, R12SO2, CF3S, and CF3SO2;

R8 and R9 are each independently selected from H or optionally substituted C1-10 alkyl;

R10, R11, R12, and R13 are each independently selected from H or optionally substituted C1-10 alkyl;

and wherein R1 and R8 may be joined to form a cyclic ring,

or a pharmaceutically acceptable ester, amide, salt, solvate, prodrug, or isomer thereof, with the proviso that when one of R8 and R9 is CH3, then at least one of R10 and R11 is optionally substituted C3-C10 cycloalkyl.

In particular embodiments of this aspect, the therapeutic agent is a compound according to the following structure:

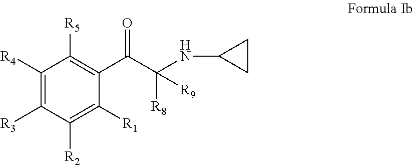

wherein

R₁-R₅ are each independently selected from H, OH, optionally substituted C1-4 alkyl, optionally substituted C1-3 alkoxy, optionally substituted C2-4 alkenyl, optionally substituted C2-4 alkynyl, halogen, amino, acylamido, CN, CF₃, NO₂, N₃, CONH₂, CO₂R₁₂, CH₂OR₁₂, NR₁₂R₁₃, NHCOR₁₂, NHCO₂R₁₂, CONR₁₁R₁₃; C1-3 alkylthio, R₁₂SO, R₁₂SO₂, CF₃S, and CF₃SO₂;

R₈ and R₉ are each independently selected from H or optionally substituted C1-10 alkyl;

R₁₂ and R₁₃ are each independently selected from H or optionally substituted C1-10alkyl; and wherein

R₁ and R₈ may be joined to form a cyclic ring,

or a pharmaceutically acceptable ester, amide, salt, solvate, prodrug, or isomer thereof.

In particular embodiments of this aspect, the therapeutic agent is a compound according to the structure:

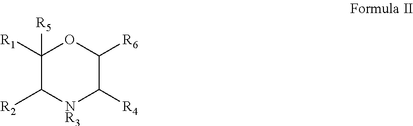

wherein

(a) R1 is optionally substituted aryl (e.g., naphthyl or phenyl);

(b) R2 is H or optionally substituted C1-3 alkyl;

(c) R3 is H, optionally substituted C1-3 alkyl, or benzyl;

(d) R4 is H or optionally substituted C1-3 alkyl;

(e) R5 is H or OH; and

(f) R6 is H or optionally substituted C1-3 alkyl;

with the proviso that when R2 is CH3 and R1 is phenyl, then

(i) the phenyl ring of R1 is substituted with one or more substituents; or

(ii) R3 is substituted C1 alkyl or optionally substituted C2-C3 alkyl, or

(iii) one or more of R4, R5, and R6 is not H, or a combination of two or more of (a) through (c);

or a pharmaceutically acceptable ester, amide, salt, solvate, prodrug, or isomer thereof.

In particular embodiments of this aspect, the therapeutic agent is a compound according to the structure:

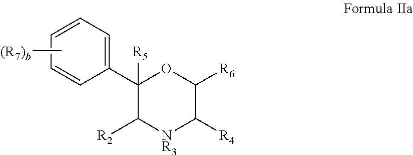

wherein

each R7 represents a substituent independently selected from the group consisting of OH, optionally substituted C1-4 alkyl, optionally substituted C1-4 alkoxy, optionally substituted C2-4 alkenyl, optionally substituted C2-4 alkynyl, Cl, F, I, acylamido, CN, CF3, N3, CONH2, CO2R12, CH2OH, CH2OR12, NHCOR12, NHCO2R12, CONR12R13, C1-3 alkylthio, R12SO, R12SO2, CF3S, and CF3SO2,

wherein R12 and R13 are each independently selected from H or optionally substituted C1-10 alkyl; and

b is an integer from 0-5;

with the proviso that when R2 is CH3, then b is an integer from 1-5 and the phenyl is trans to R2,

or a pharmaceutically acceptable ester, amide, salt, or solvate thereof.

In particular embodiments of this aspect, the therapeutic agent is a compound according to the structure:

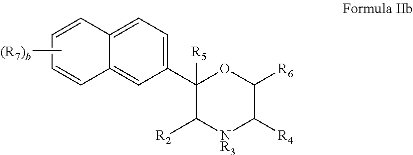

wherein

R2 is H or optionally substituted C1-3 alkyl;

R3 is H, optionally substituted C1-3 alkyl, or benzyl;

R4 is H or optionally substituted C1-3 alkyl;

R5 is H or OH;

R6 is H or optionally substituted C1-3 alkyl;

each R7 represents a substituent independently selected from the group consisting of OH, optionally substituted C1-4 alkyl, optionally substituted C1-3 alkoxy, optionally substituted C2-4 alkenyl, optionally substituted C2-4 alkynyl, halogen, amino, acylamido, CN, CF3, NO2, N3, CONH2, CO2R12, CH2OH, CH2OR12, NR12R13, NHCOR12, NHCO2R12, CONR12R13, C1-3 alkylthio, R12SO, R12SO2, CF3S, and CF3SO2; and

c is an integer from 0-7,

or a pharmaceutically acceptable ester, amide, salt, solvate, prodrug, or isomer thereof.

In particular embodiments of this aspect, the therapeutic agent is a compound according to the structure:

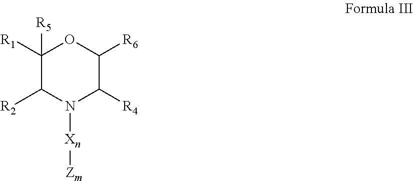

wherein

R₁, R₂, R₄, R₅, and R₆ are the same as indicated above for Formula I;

X is a chemical moiety, wherein each X may be the same or different;

n is an integer from 0 to 50, preferably 1 to 10;

Z is a chemical moiety that acts as an adjuvant, wherein each Z may be the same or different, and wherein each Z is different from at least one X; and

m is an integer from 0 to 50.

In particular embodiments of this aspect, the therapeutic agent is a compound according to the structure:

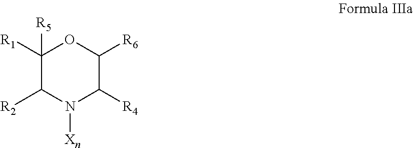

wherein

R1, R2, R4, R5, and R6 are the same as indicated above for Formula I;

X is a chemical moiety, wherein each X may be the same or different;

n is an integer from 0 to 50, preferably 1 to 10;

Z is a chemical moiety that acts as an adjuvant, wherein each Z may be the same or different, and wherein each Z is different from at least one X; and

m is an integer from 0 to 50.

In particular embodiments of this aspect, the therapeutic agent is a compound according to the structure:

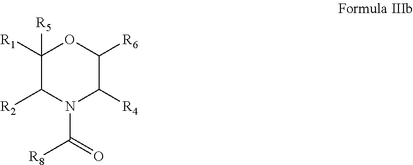

wherein

R1, R2, R4, R5, and R6 are the same as indicated above for Formula I;

R8 is optionally substituted C1-10 alkyl, optionally substituted C1-10 alkoxy, optionally substituted phenyl, optionally substituted benzyl, or optionally substituted pyridyl,

X is a chemical moiety, wherein each X may be the same or different;

n is an integer from 0 to 50, preferably 1 to 10;

Z is a chemical moiety that acts as an adjuvant, wherein each Z may be the same or different, and wherein each Z is different from at least one X; and

m is an integer from 0 to 50.

In another aspect, the therapeutic agent does not activate the 5-HT2B receptor. In alternate embodiments of that aspect, the therapeutic agent is an antagonist, i.e., a compound that blocks the activity of agonists, or it is an inverse antagonist, i.e., a compound which decreases basal activity of the receptor, or it is a neutral antagonist, i.e., a compound which blocks the binding of an agonist, of the 5-HT2B receptor. Exemplary embodiments of this aspect include but are not limited to compounds 1, 2, 24, 41, 50, 52, 56, 58, 65, 66, 68, 69, 81, 83, 86, 93, 98, 103, 105, 106, 109, 112, 114, 117, 124, 127, and 141, as disclosed in Appendix 1 herein.

The disclosure further provides methods of preventing, treating or ameliorating one or more symptoms of a disease or disorder in a patient diagnosed with that disease or disorder. In one embodiment of this aspect, the patient has been diagnosed with a seizure disorder. In further embodiments, the seizure disorder is a form of intractable epilepsy, such as Dravet syndrome, Lennox-Gastaut syndrome, Doose syndrome, and West syndrome, and other forms of refractory epilepsy. In another embodiment, the symptom is a seizure, more particularly status epilepticus. In another embodiment, the disclosure provides methods of preventing, or reducing the incidence of Sudden Death in Epilepsy (SUDEP) in a population of patients. In another embodiment, the patient is obese.

The disclosure further provides pharmaceutical compositions comprising one or more of the therapeutic agents disclosed herein for use in the methods of the invention. In some embodiments, the pharmaceutical compositions are formulations adapted to one or more dosage forms comprising an oral dosage form, an intravenous dosage form, rectal dosage form, subcutaneous dosage form, and a transdermal dosage form. In particular embodiments, the oral dosage forms are selected from the group consisting of a liquid, a suspension, a tablet, a capsule, a lozenge, and a dissolving strip. In one embodiment, the transdermal dosage form is a patch.

In another aspect, the disclosure provides a kit comprising a therapeutic agent as used in any of the methods disclosed herein, and instructions for use.

As shown above and as will be recognized by others skilled in the art, the therapeutic agents provide the important advantage that they are more effective and/or exhibit an improved safety profile as compared to fenfluramine or to other therapeutic agents and methods currently known in the art.

These and other objects, advantages, and features of the invention will become apparent to those persons skilled in the art upon reading the details of the therapeutic agents and methods of using the same as are more fully described below.

### BRIEF DESCRIPTION OF THE SEVERAL VIEWS OF THE DRAWINGS

The invention is best understood from the following detailed description when read in conjunction with the accompanying drawings. Included in the drawings are the following figures:

FIGS. 1A and 1B present, in table form, data demonstrating the inhibitory effects of test substances on radioligand binding to each of a set of 47 receptors, which data was obtained from the competitive binding assays described in Example 1.

FIG. 2 presents, in table form, the IC₅₀ values calculated for racemic fenfluramine, racemic norfenfluramine, and positive controls to selected receptors, as described in Example 2.

FIG. 3 presents Ki values calculated for racemic fenfluramine, racemic norfenfluramine, and positive controls, as described in Example 2.

FIG. 4 presents, in table form, the inhibitory effects of racemic fenfluramine and norfenfluramine, and their stereoisomers relative to positive controls, expressed as % inhibition, as described in Example 3.

FIG. 5 consists of FIGS. 5A and 5B present, in table form, the Ki values calculated for binding of fenfluramine and fenfluramine, their stereoisomers, and positive controls, as described in Example 3.

FIG. 6 presents, in table form, the test compound batch numbers used in the cellular and nuclear receptor function assays described in Example 4.

FIG. 7 presents, in table form, the experimental conditions used for the cellular and nuclear receptor function assays described in Example 4A, Example 4B, and Example 4C.

FIG. 8 presents, in table form, EC50 and IC₅₀ values calculated for stereoisomers of fenfluramine and norfenfluramine and positive controls, determined in the cellular and nuclear receptor function assays described in Example 4.

FIG. 9 presents, in table form, the experimental conditions used in the sigma receptor tissue bioassay described in Example 6, and the results of those experiments.

FIG. 10 presents, in table form, the compositions of recording solutions used for Nav1.1, 1.2, 1.3, 1.4, 1.5, 1.6, 1.7, and 1.8 in the Ion channel profiling experiments detailed in Example 5.

FIG. 11 presents the Ion flux protocol used for Nav1.8 in the ion channel profiling experiments described in Example 5.

FIG. 12 presents the ion flux protocol used in the ion channel profiling experiment described in Example 5.

FIG. 13 is a table showing the results from the Nav1.5 ion-channel profiling experiments described in Example 5. Results are expressed as normalized percentage inhibition of peak current values.

FIG. 14 consists of FIGS. 14A, 15B and 15C, wherein FIG. 14A shows a generic structure encompassing describing a series of N-alkylpropiophenones and a table listing 16 exemplary compounds encompassed by that structure, as reported in Blough et al. in ACS Med Chem Lett 2014 5 623-627. The table includes the following information for each compound: a PAL # (phenyl amine library number) and a compound number (“compd”), which are both proprietary identification numbers; the chemical formulas and specific functional groups corresponding to the functional groups designated Z, R1, R2, X, and Y, IC₅₀ and release eC50 values, and effects on transmitter uptake and release by the dopamine, serotonin, and norepinephrine. FIG. 14B shows molecular structures corresponding to the exemplary compounds listed in the table of FIG. 14A, and FIG. 14C shows synthetic synthesis schemes for making the exemplary compounds.

FIG. 15 presents, in tabular form, an overview of the assays described in Example 4, including the receptor, assay and assay format, cell line, plating density, reference agonist, reference antagonist, and concentrations used for stimulated controls (agonist assays) and agonist induction (antagonist assays).

FIG. 16 is a bar graph showing the effects of fenfluramine (FA) on decreasing epileptiform behavior in homozygous scn1Lab−/− mutant zebrafish larvae (HO), as described in Example 8A. ***p<0.001 vs. HO VHC; n=16-30 ZF larvae for all experimental conditions.

FIG. 17 consists of FIG. 17A and FIG. 17B which are each bar graphs showing the effects of fenfluramine (FA) on epileptiform brain activity in homozygous scn1Lab−/− mutant zebrafish larvae (HO) during a 10-minute recording period following fenfluramine treatment, as described in Example 8A. FIG. 19A shows fenfluramine's effects on the frequency of epileptiform events. FIG. 19B shows fenfluramine's effects on cumulative duration (msec) of epileptiform events. *p<0.05, **p<0.01 and ***p<0.001 vs. HO VHC; n=8-18 ZF larvae for all experimental conditions.

FIG. 18 is a bar graph showing the effects of fenfluramine (FA) on PTZ-induced seizures in wild-type zebrafish (ZF) larvae, determined using the behavioral (locomotor) assay described in Example 7B. ***p<0.001 vs. VHC+PTZ; n=24-36 ZF larvae for all experimental conditions.

FIG. 19 consists of FIGS. 19A and 19B which show the effects of Fenfluramine (FA) treatment in 6-Hz mice, as described in Example 7C. FIG. 19A is a bar graph showing the percentage of animals protected for mice treated with vehicle, with 20 mg FA, and with 5 mg/kg FA. FIG. 19B shows the effects on seizure duration. **p<0.01 and ***p<0.001 vs. VHC-injected; n=6-10 NMRI mice for all experimental conditions.

FIG. 20 shows a schematic isobologram plot used in the isobologram analysis described in Example 7A and Example 7B.

FIG. 21 is a bar graph showing the antidepressant-like effect of 8-OH-DPAT and/or igmesine in the forced swim test (FST) described in Example 73. *p<0.05, **p<0.01, ***p<vs. V-treated group; Dunnett's test.

FIG. 22 shows the Combination Index calculated for Igmesine and 8-OH-DPAT using FST data, as described in Example 7(A).

FIG. 23 consists of FIGS. 23A, 23B, and 23C which are each bar graphs showing the dose-response effect of fenfluramine on dizocilpine-induced alteration spontaneous alternation response in the Y-maze in mice. 23A plots alternation performances, 23B plots total number of arm entries, and 23C plots the combined effects of fenfluramine with the sigma-1 receptor agonist PRE-084. **p<0.01, ***p<0.001 vs. V-treated group; ##p<0.01, ###p<0.001 vs. Dizocilpine-treated group; Dunnett's test. °p<0.05, °°°p<0.001; Student's t-test.

FIG. 24 shows the Combination Index calculation for Igmesine and 8-OH-DPAT using spontaneous alternation data, as described in Example 7(B).

FIG. 25 consists of FIGS. 25A, 25B, and 25C are bar graphs showing dose-response effects of fenfluramine on dizocilpine-induced alteration of passive avoidance response in mice. FIG. 25A shows fenfluramine's effects on step-through latency. FIG. 25B show fenfluramine's effects on escape latency. FIG. 25C shows the combined effects of fenfluramine and the sigma-1 receptor agonist PRE-084 using the step-through latency parameter. **p<0.01, ***p<0.001 vs. V-treated group; ##p<0.01, ###p<0.001 vs. Dizocilpine-treated group; Mann-Whitney's test.

FIG. 26 shows the Combination Index calculations for fenfluramine and PRE-084 using passive avoidance data, as described in Example 7(B).

FIG. 27 is a dose-response curve plotting data from the dose-response study described in Example 9, and showing the effects of increasing fenfluramine dosage on the susceptibility of DBA/1 mice to seizure-induced respiratory arrest (S-IRA).

FIG. 28 is a dose-response curve plotting data from the dose-response study described in Example 9, and showing the effects of increasing fenfluramine dosage on the susceptibility of DBA/1 mice to audiogenic seizures (AGSz).

FIG. 29 plots data from the time-course study described in Example 9, and shows the effects fenfluramine, administered at 10 mg/kg or 15 mg/kg, on the susceptibility of DBA/1 mice to S-IRA over a 72 hour period.

FIG. 30 plots data from the time-course study described in Example 9, and shows the effects fenfluramine, administered at 10 mg/kg or 15 mg/kg, on the susceptibility of DBA/1 mice to audiogenic seizures over a 72 hour period.

### SUPPLEMENTAL MATERIALS

Appendix 1 provides, in tabular form, exemplary embodiments of the invention described and claimed herein and forms a part of this application.

### DETAILED DESCRIPTION OF THE INVENTION

Before the present compositions and methods are described, it is to be understood that this invention is not limited to the particular formulations and methods described, as such can, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges can independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those include limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims the singular forms “a,” “an,” and “the” include plural referents unless the context clearly dictates otherwise.

Thus, for example, reference to “a formulation” includes a plurality of such formulations and reference to “the method” includes reference to one or more methods and equivalents thereof known to those skilled in the art, and so forth.

The publications discussed herein are provided solely for their disclosure prior to the filing of the present application. Nothing herein is to be construed as an admission that the present invention is not entitled to antedate such publication by virtue of prior invention. Further, the dates of publication provided may be different from the actual publication dates which may need to be independently confirmed.

### OVERVIEW OF THE INVENTION

Previously, fenfluramine's activity and therefore its therapeutic effects were thought to be mediated by its activity at certain serotonergic receptor subtypes and neurotransmitter transporter proteins.

The inventors' work more fully elucidates fenfluramine's mechanism of action. Without being bound by theory, it unexpectedly reveals that fenfluramine is active at multiple receptors. Surprisingly, and without being bound by theory, their data reveals that, in addition to binding 5-HT receptors, particularly 5-HT1A, fenfluramine also binds the β-2 adrenergic receptor, the Muscarinic M1 receptor, the Nav 1.5 sodium channel subunit, and the Sigma-1 receptor. See Example 1, Example 2, Example 3, and Example 4 and related figures. Further, and without being bound by theory, they have surprisingly discovered that fenfluramine is active as a positive allosteric modulator (PAM) of the Sigma 1 receptor. See Example 7 and related figures.

Further, the inventors have confirmed fenfluramine's efficacy in reducing seizures in a zebrafish genetic model of Dravet syndrome. Further, they have expanded that understanding, by unexpectedly discovering that fenfluramine is also effective in reducing seizures in a 6 Hz mouse model of refractory epilepsy. See Example 8 and related figures.

Finally, the inventors have surprisingly discovered that, in addition to its efficacy in reducing seizure activity in patients diagnosed with Dravet syndrome as well as animal models of that disease, fenfluramine is also effective in reducing seizures in a mouse model of seizure-induced respiratory arrest and audiogenic seizures in DBA/1 mice. See Example 9 and related figures.

### SPECIFIC ASPECTS OF THE INVENTION

Provided are therapeutic agents that are useful in preventing, treating, or ameliorating symptoms associated with a disease or disorder in a patient diagnosed with the disease or disorder, including but not limited to patients diagnosed with refractory epilepsy, including but not limited to Dravet syndrome, Lennox-Gastaut syndrome, Doose syndrome, and West syndrome, and other refractory epilepsies. Also provided are methods of preventing, treating or ameliorating symptoms such as seizures and seizure-induced respiratory arrest (S-IRA) leading to sudden unexpected death in epilepsy (SUDEP) associated with a disease or disorder in a patient diagnosed with that disease or disorder, and pharmaceutical compositions and formulations comprising those agents that are useful in practicing the methods of the invention.

### Therapeutic Agents

The inventors have made the surprising discovery that certain therapeutic agents are useful in treating diseases or disorders, including but not limited to diseases or disorders associated with intractable seizures, seizure-induced respiratory arrest (S-IRA) and sudden unexplained death in epilepsy (SUDEP). Thus, in accordance with one aspect of the invention, the disclosure provides therapeutic agents that are useful in treating patients diagnosed with a disease or disorder and/or in preventing or ameliorating symptoms of those diseases or disorders exhibited by the patient.

### Target Binding

In one embodiment of that aspect, the therapeutic agent binds one or more targets selected from the group consisting of a receptor protein, a sodium channel subunit, a chaperone protein, and a neurotransmitter transporter protein.

### Receptor Protein Targets

In one embodiment of this aspect, the therapeutic agent binds a receptor protein selected from the group consisting of a 5-HT receptor, such as the 5-HT1A receptor, the 5-HT1D receptor, the 5-HT1E receptor, the 5-HT2A receptor, the 5-HT2C receptor, the 5-HT5A receptor, and the 5-HT7 receptor. In a preferred embodiment, the therapeutic agent binds a receptor protein selected from the group consisting of a 5-HT receptor, such as the 5-HT1A receptor, the 5-HT1D receptor, the 5-HT1E receptor, the 5-HT2A receptor, the 5-HT2C receptor, the 5-HT5A receptor, and the 5-HT7 receptor. In one exemplary embodiment, the therapeutic agent binds the 5-HT1A receptor. In another exemplary embodiment, the therapeutic agent binds the 5-HT1D receptor. In another exemplary embodiment, the therapeutic agent binds the 5-HT2A receptor. In another exemplary embodiment, the therapeutic agent binds the 5-HT2C receptor.

In another embodiment, the therapeutic agent binds an adrenergic receptor, such as the beta-1 receptor or the beta-2 adrenergic receptor. In a preferred embodiment, the therapeutic agent binds the beta-2 adrenergic receptor.

In other embodiment, the therapeutic agent binds a muscarinic acetylcholine receptor selected from the group consisting of the M1 muscarinic acetylcholine receptor the M2 muscarinic acetylcholine receptor, the M3 muscarinic acetylcholine receptor, the M4 muscarinic acetylcholine receptor, and the M5 muscarinic acetylcholine receptor. In an exemplary embodiment, the therapeutic agent binds the muscarinic M1 acetylcholine receptor.

### Sodium Channel Subunit Targets

In another embodiment of that aspect, the disclosure provides a therapeutic agent that binds to a sodium channel receptor, such as, for example, one or more of the Nav1.1 sodium channel, the Nav1.2 sodium channel, the Nav1.3 sodium channel, the Nav1.4 sodium channel, the Nav1.5 sodium channel, the Nav1.6 sodium channel, and/or the Nav1.7 sodium channel.

### Chaperone Protein Targets

In another embodiment of that aspect, the disclosure provides a therapeutic agent that binds to a chaperone protein such as, for example, the sigma-1 receptor or the sigma-2 receptor. In one exemplary embodiment, the disclosure provides a therapeutic agent that binds to the sigma-1 receptor. In another exemplary embodiment, the disclosure provides a therapeutic agent that binds to the sigma-1 receptor.

### Neurotransmitter Transporter Protein Targets

In another embodiment of that aspect, the disclosure provides a therapeutic agent that binds to one or more neurotransmitter transport proteins selected from the group consisting of a serotonin transporter (SERT), a dopamine transporter (DAT), and a norepinephrine transporter (NET). In one exemplary embodiment, the therapeutic agent binds a SERT protein. In another exemplary embodiment, the therapeutic agent binds a NET protein. In another exemplary embodiment, the therapeutic agent binds a DAT protein.

### Binding of Single or Multiple Targets

In some embodiments, the therapeutic agents provided by the disclosure can bind one or more targets, for example, two or more targets, three or more targets, four or more targets, five or more targets, or more.

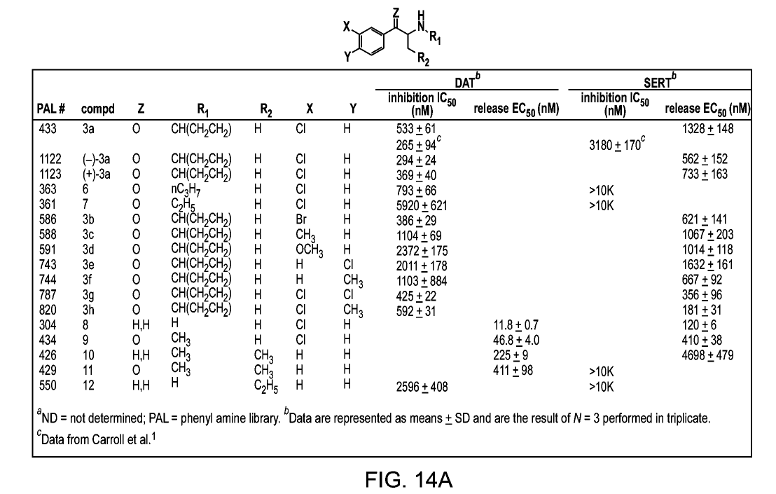

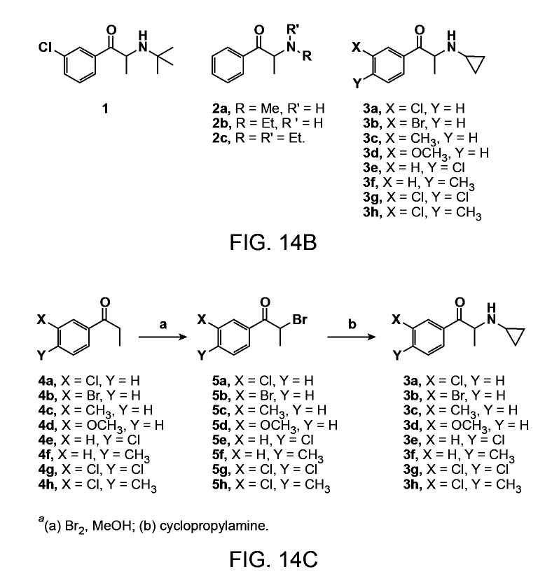

For example, the disclosure provides therapeutic agents that bind to two or more neurotransmitter transporters. Exemplary embodiments include but are not limited to PAL 433, PAL 1122, PAL 1123, PAL 363, PAL 361, PAL 586, PAL 588, PAL 591, PAL 743, PAL 744, PAL 787, PAL 820, PAL 304, PAL 434, PAL 426, PAL 429, and PAL 550 as shown in FIG. 14A. In a preferred embodiment, the therapeutic agent is PAL820. In another preferred embodiment, the therapeutic agent is PAL787.

In preferred embodiments, the therapeutic agent binds to the sigma-1 receptor and one or more 5-HT receptor, for example, the 5-HT1A receptor, the 5-HT1D receptor, the 5-HT1E receptor, the 5-HT2A receptor, the 5-HT2C receptor, the 5-HT5A receptor, and/or the 5-HT7 receptor. In preferred embodiments, the therapeutic agent binds to the sigma-1 receptor and one or more receptor protein selected from the group consisting of the 5-HT1A receptor, the 5-HT1D receptor, the 5-HT2A receptor, and/or the 5-HT2C receptor. In one preferred embodiment, the therapeutic agent binds to the sigma-1 receptor and the 5-HT1A receptor. In another preferred embodiment, the therapeutic agent binds to the sigma-1 receptor and the 5-HT1D receptor. In another preferred embodiment, the therapeutic agent binds to the sigma-1 receptor and the 5-HT2A receptor. In another preferred embodiment, the therapeutic agent binds to the sigma-1 receptor and the 5-HT2C receptor.

### Functional Activity

In accordance with one aspect of the invention, the disclosure provides therapeutic agents that are active at one or more targets selected from the group consisting of a receptor protein, a sodium channel subunit protein, a chaperone protein, and a neurotransmitter transport protein. The terms “active” or “activity” as used herein to mean an effect on cell, nuclear, or tissue function, and is intended to encompass agonist activity, inverse agonist activity, antagonist activity, synergy, allosteric agonism, allosteric modulation, including positive, negative and neutral allosteric modulation, ago-allosteric modulation, including positive, negative, and neutral ago-allosteric modulation, and ligand trapping.

### Receptor Activity

In one embodiment of that aspect, the therapeutic agent is active at one or more 5-HT receptor proteins selected from the group consisting of the 5-HT1A receptor, the 5-HT1D receptor, the 5-HT2A receptor, and the 5-HT2C receptor.

### Sodium Channel Subunit Activity

In another exemplary embodiment, the therapeutic agents are active at a sodium channel subunit selected from the group consisting of the Nav 1.1 subunit, the Nav 1.2 sodium channel subunit, the Nav 1.3 sodium channel subunit, the Nav 1.4 sodium channel subunit, the Nav1.5 sodium channel subunit, the Nav 1.6 subunit, the Nav 1.7 subunit, and the Nav 1.8 subunit.

### Chaperone Protein Activity

In another embodiment, the therapeutic agent is active at a chaperone protein. Exemplary embodiments include but are not limited to, the sigma-1 receptor and the sigma-2 receptor. In a preferred embodiment, the therapeutic agent is active at the sigma-1 receptor. In a preferred embodiment, the therapeutic agent is a positive allosteric modulator of the sigma-1 receptor.

### Neurotransmitter Transport Protein Activity

In another embodiment of that aspect, the disclosure provides a therapeutic agent that is active at one or more intracellular neurotransmitter transport proteins selected from the group consisting of a serotonin transport protein (SERT), a norepinephrine transport protein (NET), and a dopamine transport protein (DAT). In some embodiments, the therapeutic agent acts to inhibit neurotransmitter reuptake, for example by blocking binding of the neurotransmitter to the transporter or by preventing conformational changes which transporter activity. In some embodiments, the therapeutic agent stimulates neurotransmitter release, for example by acting as a transporter substrate.

### Therapeutic Agents Active at Multiple Targets

The disclosure further provides therapeutic agents that are active one or more targets, for example, two or more targets, three or more targets, four or more targets, five or more targets, or more.

For example, in one embodiment, the disclosure provides therapeutic agents that are active at two or more neurotransmitter transporters. In this regard, the inventors have made the surprising discovery that certain compounds which act on more than one biogenic amine transporter (BAT) are useful in treating patients diagnosed with a seizure disease or disorder, including patients diagnosed with intractable epilepsy syndromes.

Thus, in one embodiment, the therapeutic agents provided by the disclosure herein are functional hybrids that act on two or more neurotransmitter transport proteins selected from the group consisting of the SERT protein, the DAT protein, and the NET protein, to block neurotransmitter uptake or stimulate neurotransmitter release or both. For example, the therapeutic agents are functional hybrids which act on the DAT protein to block uptake of dopamine and also acts on the SERT protein to stimulate release of serotonin.

In one embodiment, therapeutic agents which find use in the methods of the present invention are bupropion structural analogs capable of inhibiting the reuptake of one or more monoamines, according to the following structure:

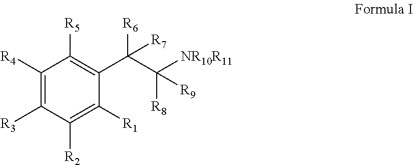

wherein R1-R5 are each independently selected from H, OH, optionally substituted C1-4 alkyl, optionally substituted C1-3 alkoxy, optionally substituted C2-4 alkenyl, optionally substituted C2-4 alkynyl, halogen, amino, acylamido, CN, CF3, NO2, N3, CONH2, CO2R12, CH2OR12, NR12R13, NHCOR12, NHCO2R12, CONR12R13; C1-3 alkylthio, R12SO, R12SO2, CF3S, and CF3SO2;

R6 and R7 are each independently selected from H or optionally substituted C1-10alkyl, or R6 and R7 together constitute ═O or ═CH2;

R8 and R9 are each independently selected from H or optionally substituted C1-10alkyl;

R10, R11, R12, and R13 are each independently selected from H or optionally substituted C1-10 alkyl;

and wherein R1 and R8 may be joined to form a cyclic ring; or a pharmaceutically acceptable ester, amide, salt, solvate, prodrug, or isomer thereof,

with the proviso that when one of R8 and R9 is CH3, then at least one of R10 and R11 is optionally substituted C3-C10 cycloalkyl.

In particular embodiments, therapeutic agents according to the following structure are useful in the methods disclosed herein:

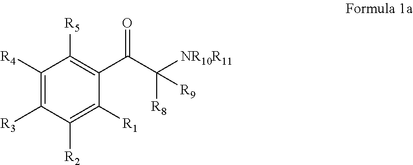

wherein

R1-R5 are each independently selected from H, OH, optionally substituted C1-4 alkyl, optionally substituted C1-3 alkoxy, optionally substituted C2-4 alkenyl, optionally substituted C2-4 alkynyl, halogen, amino, acylamido, CN, CF3, NO2, N3, CONH2, CO2R12, CH2OR12, NR12R13, NHCOR12, NHCO2R12, CONR12R13; C1-3 alkylthio, R12SO, R12SO2, CF3S, and CF3SO2;

R8 and R9 are each independently selected from H or optionally substituted C1-10 alkyl;

R10, R11, R12, and R13 are each independently selected from H or optionally substituted C1-10 alkyl;

and wherein R1 and R8 may be joined to form a cyclic ring,

or a pharmaceutically acceptable ester, amide, salt, solvate, prodrug, or isomer thereof, with the proviso that when one of R8 and R9 is CH3, then at least one of R10 and R11 is optionally substituted C3-C10 cycloalkyl.

In further particular embodiments, the methods disclosed herein employ compounds according to the following structure:

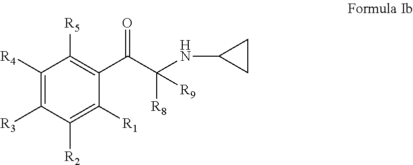

wherein

R₁-R₅ are each independently selected from H, OH, optionally substituted C1-4 alkyl, optionally substituted C1-3 alkoxy, optionally substituted C2-4 alkenyl, optionally substituted C2-4 alkynyl, halogen, amino, acylamido, CN, CF₃, NO₂, N₃, CONH₂, CO₂R₁₂, CH₂OR₁₂, NR₁₂R₁₃, NHCOR₁₂, NHCO₂R₁₂, CONR₁₁R₁₃; C1-3 alkylthio, R₁₂SO, R₁₂SO₂, CF₃S, and CF₃SO₂;

R₈ and R₉ are each independently selected from H or optionally substituted C1-10 alkyl;

R₁₂ and R₁₃ are each independently selected from H or optionally substituted C1-10alkyl; and

wherein R₁ and R₈ may be joined to form a cyclic ring,

or a pharmaceutically acceptable ester, amide, salt, solvate, prodrug, or isomer thereof.

In another embodiment, therapeutic agents which find use in the methods of the present invention are compounds capable of functioning as releasers and/or uptake inhibitors or one or more monoamine neurotransmitters, including dopamine, serotonin, and norepinephrine, wherein the therapeutic agent is a morpholine compound according to the structure:

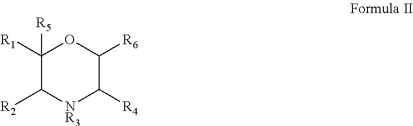

wherein

R₁ is optionally substituted aryl (e.g., naphthyl or phenyl);

R₂ is H or optionally substituted C1-3 alkyl;

R₃ is H, optionally substituted C1-3 alkyl, or benzyl;

R₄ is H or optionally substituted C1-3 alkyl;

R₅ is H or OH; and

R₆ is H or optionally substituted C1-3 alkyl;

with the proviso that when R₂ is CH₃ and R₁ is phenyl, then (a) the phenyl ring of R₁ is substituted with one or more substituents; or (b) R₃ is substituted C1 alkyl or optionally substituted C2-C3 alkyl, or (c) one or more of R₄, R₅, and R₆ is not H, or a combination of two or more of (a) through (c);

or a pharmaceutically acceptable ester, amide, salt, solvate, prodrug, or isomer thereof.

In one particular embodiment, the compound of Formula II can be represented by Formula IIa.

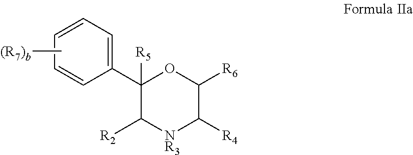

wherein:

R2 is H or optionally substituted C1-3 alkyl;

R3 is H, optionally substituted C1-3 alkyl, or benzyl;

R4 is H or optionally substituted C1-3 alkyl;

R5 is H or OH;

R6 is H or optionally substituted C1-3 alkyl;

each R7 represents a substituent independently selected from the group consisting of OH, optionally substituted C1-4 alkyl, optionally substituted C1-4 alkoxy, optionally substituted C2-4 alkenyl, optionally substituted C2-4 alkynyl, halogen, amino, acylamido, CN, CF3, NO2, N3, CONH2, CO2R12, CH2OH, CH2OR12, NR12R13, NHCOR12, NHCO2R12, CONR12R13, C1-3 alkylthio, R12SO, R12SO2, CF3S, and CF3SO2, wherein R12 and R13 are each independently selected from H or optionally substituted C1-10 alkyl;

b is an integer from 0-5; and

with the proviso that when R2 is CH3, then (a) b is an integer from 1-5, or (b) R3 is substituted C1 alkyl or optionally substituted C2-C3 alkyl, or (c) one or more of R4, R5, and R6 is not H, or a combination of two or more of (a) through (c), or a pharmaceutically acceptable ester, amide, salt, solvate, prodrug, or isomer thereof.

In another particular embodiment, the compound of Formula II can be represented by Formula IIb:

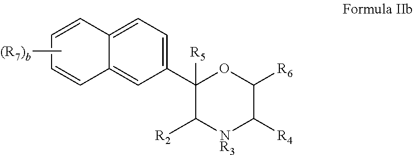

wherein

R₂ is H or optionally substituted C1-3 alkyl;

R₃ is H, optionally substituted C1-3 alkyl, or benzyl;

R₄ is H or optionally substituted C1-3 alkyl;

R₅ is H or OH;

R₆ is H or optionally substituted C1-3 alkyl;

each R₇ represents a substituent independently selected from the group consisting of OH, optionally substituted C1-4 alkyl, optionally substituted C1-3 alkoxy, optionally substituted C2-4 alkenyl, optionally substituted C2-4 alkynyl, halogen, amino, acylamido, CN, CF₃, NO₂, N₃, CONH₂, CO₂R₁₂, CH₂OH, CH₂OR₁₂, NR₁₂R₁₃, NHCOR₁₂, NHCO₂R₁₂, CONR₁₂R₁₃, C1-3 alkylthio, R₁₂SO, R₁₂SO₂, CF₃S, and CF₃SO₂; and

c is an integer from 0-7,

or a pharmaceutically acceptable ester, amide, salt, solvate, prodrug, or isomer thereof.

In some embodiments of the present invention, therapeutically inactive prodrugs are provided. Prodrugs are compounds which, when administered to a mammal, are converted in whole or in part to a compound of the invention. In most embodiments, the prodrugs are pharmacologically inert chemical derivatives that can be converted in vivo to the active drug molecules to exert a therapeutic effect. Any of the compounds described herein can be administered as a prodrug to increase the activity, bioavailability, or stability of the compound or to otherwise alter the properties of the compound. Typical examples of prodrugs include compounds that have biologically labile protecting groups on a functional moiety of the active compound. In preferred embodiments, the nitrogen atom of the morpholine in any of Formulas II, Formula IIa, and Formula IIb above is functionalized with such a chemical moiety. Prodrugs include, but are not limited to, compounds that can be oxidized, reduced, aminated, deaminated, hydroxylated, dehydroxylated, hydrolyzed, dehydrolyzed, alkylated, dealkylated, acylated, deacylated, phosphorylated, and/or dephosphorylated to produce the active compound.

A number of prodrug ligands are known. In general, alkylation, acylation, or other lipophilic modification of one or more heteroatoms of the compound, such as a free amine or carboxylic acid residue, may reduce polarity and allow for the compound's passage into cells. The means by which the modification of one or more heteroatoms of the compound is performed may vary, and typical methods for such modifications are familiar to one of skill in the art of organic synthesis. For example, general reaction conditions for the alkylation and acylation of heteroatoms are well known and can be modified for application to the compounds provided herein.

Prodrugs useful in methods according to the present invention can be represented by Formula III:

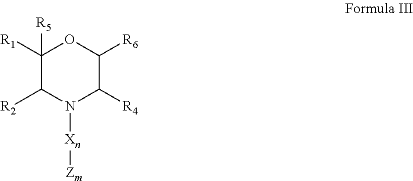

wherein

R1, R2, R4, R5, and R6 are the same as indicated above for Formula II;

X is a chemical moiety, wherein each X may be the same or different;

n is an integer from 0 to 50, preferably 1 to 10;

Z is a chemical moiety that acts as an adjuvant, wherein each Z may be the same or different, and wherein each Z is different from at least one X; and

m is an integer from 0 to 50.

In some embodiments, X may be alkyl. In some embodiments, when R2 is CH3, R1 is phenyl, R4-R6 are H, n=1, and m=0, X is not CH3. In some, but not all, embodiments of Formula IV, when R1 is phenyl, the phenyl ring is substituted with one or more substituents and/or one or more of R4, R5, and R6 is not H.

The chemical moiety constituting X can be any chemical moiety that, while bound to the compound, decreases the pharmacological activity of the compound in comparison to the free compound. In some embodiments, X is any pharmaceutically acceptable chemical moiety which, when the prodrug is administered in vivo, is cleaved in whole or in part to provide a free amine on the morpholine ring. Exemplary chemical moieties include, but are not limited to, peptides, carbohydrates (including sugars), lipids, nucleosides, nucleic acids, and vitamins, aryl groups; steroids; 1,2-diacylglycerol; alcohols; optionally substituted acyl groups (including lower acyl); optionally substituted alkyl groups (including lower alkyl); sulfonate esters (including alkyl or arylalkyl sulfonyl, such as methanesulfonyl and benzyl, wherein the phenyl group is optionally substituted with one or more substituents as provided in the definition of an aryl given herein); optionally substituted arylsulfonyl groups; lipids (including phospholipids); phosphotidylcholine; phosphocholine; amino acid residues or derivatives; amino acid acyl residues or derivatives; cholesterols; or other pharmaceutically acceptable leaving groups which, when administered in vivo, provide the free amine and/or carboxylic acid moiety. Peptides include dipeptides, tripeptides, oligopeptides, and polypeptides.

In particular embodiments, prodrugs useful in the present invention can be represented by Formula IIIa

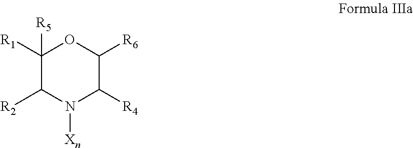

wherein the substituents are the same as those indicated for Formula IV.

In particular embodiments, prodrugs of the present invention can be represented by Formula IIIb:

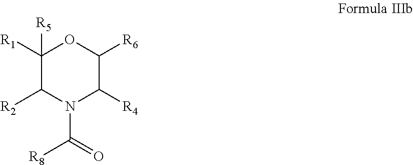

wherein the substituents are the same as those indicated for Formula III, except that:

R8 is optionally substituted C1-10 alkyl, optionally substituted C1-10 alkoxy, optionally substituted phenyl, optionally substituted benzyl, or optionally substituted pyridyl.

In an exemplary embodiment, the therapeutic agent blocks neurotransmitter reuptake and stimulate neurotransmitter release. Examples of such hybrid agents include but are not limited the compounds designated as PAL 433, PAL 1122, PAL 1123, PAL 363, PAL 361, PAL 586, PAL 588, PAL 591, PAL 743, PAL 744, PAL 787, PAL 820, PAL 304, PAL 434, PAL 426, PAL 429, and PAL 550, described in Blough et. al, ACS Med. Chem. Let. (2014), 5, 623-627, and shown in FIG. 14A herein.

Thus, in one aspect, the therapeutic agents provided by the disclosure herein are functional hybrids that act on two or more neurotransmitter transport proteins selected from the group consisting of the SERT protein, the DAT protein, and the NET protein, to block neurotransmitter uptake or stimulate neurotransmitter release or both. In one embodiment of this aspect, the therapeutic agents are functional hybrids which act on the DAT protein to block uptake of dopamine and also acts on the SERT protein to stimulate release of serotonin. In one embodiment, the compounds are N alkylpropiophenones.

In various exemplary embodiments, the N alkylpropiophenones are species of Structure II (also shown FIG. 14A):

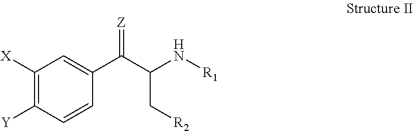

Exemplary embodiments of such hybrid agents include but are not limited to the N-alkylpropiophenones species encompassed by Structure II, including but not limited to PAL 433, PAL 1122, PAL 1123, PAL 363, PAL 361, PAL 586, PAL 588, PAL 591, PAL 743, PAL 744, PAL 787, PAL 820, PAL 304, PAL 434, PAL 426, PAL 429, and PAL 550, as shown in FIG. 14A. Preferred embodiments are PAL 787 and PAL 820. Other examples of agents which are functional hybrids are possible and are contemplated as useful in treating patients, including patients diagnosed with certain forms of epilepsy, and in seizure control.

Therapeutic Agents which are Inactive at the 5-HT2B Receptor

In preferred embodiments, the therapeutic agents disclosed herein are not active at the 5-HT2B receptor to an extent sufficient to cause adverse effects such as valvulopathy, pulmonary hypertension or other adverse effects. In alternate exemplary embodiments, the agents do not bind the 5-HT2B receptor, or are 5-HT2B antagonists, i.e., agents that block the activity of agonists, or are 5-HT2B inverse antagonists i.e., agents that decrease basal activity of the receptor, or are neutral agonists, i.e., compounds that block binding of agonists, of the 5-HT2B receptor.

Exemplary embodiments of this aspect include but are not limited to the compounds designated as 1, 2, 24, 41, 50, 52, 56, 58, 65, 66, 68, 69, 81, 83, 86, 93, 98, 103, 105, 106, 109, 112, 114, 117, 124, 127, and 141, as disclosed in Appendix 1 herein, and compounds PAL 433, PAL 1122, PAL 1123, PAL 363, PAL 361, PAL 586, PAL 588, PAL 591, PAL 743, PAL 744, PAL 787, PAL 820, PAL 304, PAL 434, PAL 426, PAL 429, and PAL 550, as shown in the table appearing in FIG. 14A.

Hybrid molecules such as are described by Formula I, Formula Ia, Formula Ib, Formula II, Formula IIa, Formula IIb, Formula III, Formula IIIa and Formula IIIb can be synthesized using methods commonly known in the art, or by synthetic methods such as are disclosed in U.S. Pat. No. 9,562,001 and in issued U.S. Pat. No. 9,617,229, which are by reference incorporated in their entirety herein.

### Screening

Therapeutic agents that are useful in the methods disclosed herein can be identified by using methods that are known in the art. For example, compounds may be screened using a high-throughput mutant zebrafish embryo assay to measure effects on epileptiform activity and locomotion. See e.g., Zhang et al., ACS Nano, 2011, 5 (3), pp 1805-1817; DOI: 10.1021/nn102734s, e-published on Feb. 16, 2011, and Example 9 herein.

### Diseases and Disorders

The therapeutic agents provided by the disclosure are useful in treating a number of diseases and disorders, and/or in reducing or ameliorating their symptoms. For example, the therapeutic agents disclosed herein are useful for treating forms of epilepsy such as Dravet syndrome, Lennox-Gastaut syndrome, Doose syndrome, West syndrome, and other refractory epilepsy syndromes, and in preventing, reducing or ameliorating their symptoms in patients diagnosed with those conditions. The therapeutic agents provided herein are also useful in preventing cognition disorders that affects learning, memory, perception, and/or problem solving, including but not limited to amnesia, dementia, and delirium.

### Methods of Use

The above-described therapeutic agents can be employed in a variety of methods. As summarized above, aspects of the method include administering a therapeutically effective amount of a therapeutic agent as described herein to treat a patient in need of treatment, for example, to a patient diagnosed with a disease or condition of interest, or to prevent, reduce or ameliorate symptoms of a disease or disorder in patients diagnosed with that disease or disorder. Examples include seizures, particularly status epilepticus, seizure-induced respiratory arrest (S-IRA), and Sudden Unexplained Death in Epilepsy (SUDEP). By “therapeutically effective amount” is meant the concentration of a compound that is sufficient to elicit the desired biological effect (e.g., treatment or prevention of epilepsy and associated symptoms and co-morbidities, including but not limited to seizure-induced sudden respiratory arrest (S-IRA). Diseases and conditions of interest include, but are not limited to, epilepsy, particularly intractable forms of epilepsy, including but not limited to Dravet syndrome, Lennox-Gastaut syndrome, Doose syndrome, West syndrome, and other refractory epilepsies, as well as other neurological related diseases, obesity, and obesity-related diseases. Also of interest is the prevention or amelioration of symptoms and co-morbidities associated with those diseases

In some embodiments, the subject method includes administering to a subject a compound to treat a neurological related disease. Neurological related diseases of interest include, but are not limited to, epilepsy, particularly severe or intractable forms of epilepsy, including but not limited to severe myoclonic epilepsy in infancy (Dravet syndrome), Lennox-Gastaut syndrome, Doose syndrome, West syndrome, and other refractory epilepsies. In some embodiments, the subject method will be protective of symptoms, including but not limited to S-IRA, SUDEP, and co-morbid conditions.

### Genetic Testing

In some cases, it can be desirable to test the patients for a genetic mutation prior to administration of some of the therapeutic agents provided by the disclosure, especially in cases where use of specific agent is contraindicated either because the agent is ineffective or because it would have undesired or serious side effects. Thus, it is in some cases desirable to test patients prior to treatment. In the case of patients having Dravet syndrome, testing can be carried out for mutations in the SCN1A (such as partial or total deletion mutations, truncating mutations and/or missense mutations e.g. in the voltage or pore regions S4 to S6), SCN1 B (such as the region encoding the sodium channel β1 subunit), SCN2A, SCN3A, SCN9A, GABRG2 (such as the region encoding the γ2 subunit), GABRD (such as the region encoding the σ subunit) and I or PCDH19 genes have been linked to Dravet syndrome.

Similarly, several reports in the literature evidence a strong, likely multifactorial genetic component for Doose syndrome (see e.g., Kelly et al., Developmental Medicine & Child Neurology 2010, 52: 988-993), and a number of mutations appear in a significant number of Doose syndrome patients, including sodium channel neuronal type 1 alpha subunit (SCN1A) mutations, sodium channel subunit beta-1 (SCN1B) and gamma-aminobutyric acid receptor, subunit gamma-2 (GABRG2) mutations; point mutations in exon 20 of SCN1A

Other genetic tests can be carried out, and can be required as a condition of treatment.

### Dosing

The different therapeutic agents disclosed herein can be dosed to patients in different amounts depending on different patient age, size, sex, condition as well as the use of different therapeutic agents.

For example, the dosing can be a daily dosing based on weight. However, for convenience the dosing amounts can be preset. In general, the smallest dose which is effective should be used for the particular patient. The patient can be dosed on a daily basis using a single dosage unit which single dosage unit can be comprised of the therapeutic agent in an amount appropriate for the particular agent. The dosage unit can be selected based on the delivery route, e.g. the dosage unit can be specific for oral delivery, transdermal delivery, rectal delivery, buccal delivery, intranasal delivery, pulmonary delivery or delivery by injection.

### Formulation

The dose of therapeutic agent administered in the methods of the present invention can be formulated in any pharmaceutically acceptable dosage form including, but not limited to oral dosage forms such as tablets including orally disintegrating tablets, capsules, lozenges, oral solutions or syrups, oral emulsions, oral gels, oral films, buccal liquids, powder e.g. for suspension, and the like; injectable dosage forms; transdermal dosage forms such as transdermal patches, ointments, creams; inhaled dosage forms; and/or nasally, rectally, vaginally administered dosage forms. Such dosage forms can be formulated for once a day administration, or for multiple daily administrations (e.g. 2, 3 or 4 times a day administration).

Particular formulations of the invention are in a liquid form. The liquid can be a solution or suspension and can be an oral solution or syrup which is included in a bottle with a pipette which is graduated in terms of milligram amounts which will be obtained in a given volume of solution. The liquid solution makes it possible to adjust the solution for small children which can be administered in increments appropriate to the particular therapeutic agent.

Administration of the subject compounds can be systemic or local. In certain embodiments, administration to a mammal will result in systemic release of a subject compound (for example, into the bloodstream). Methods of administration can include enteral routes, such as oral, buccal, sublingual, and rectal; topical administration, such as transdermal and intradermal; and parenteral administration. Suitable parenteral routes include injection via a hypodermic needle or catheter, for example, intravenous, intramuscular, subcutaneous, intradermal, intraperitoneal, intraarterial, intraventricular, intrathecal, and intracameral injection and non-injection routes, such as intravaginal rectal, or nasal administration. In certain embodiments, the subject compounds and compositions are administered orally. In certain embodiments, it can be desirable to administer a compound locally to the area in need of treatment. In some embodiments, the method of administration of the subject compound is parenteral administration. This can be achieved, for example, by local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers.

In some embodiments, the subject method includes administering to a subject an appetite suppressing amount of the subject compound to treat obesity. Any convenient methods for treating obesity can be adapted for use with the subject therapeutic agents. Any of the pharmaceutical compositions described herein can find use in treating a subject for obesity. Combination therapy includes administration of a single pharmaceutical dosage formulation which contains the subject compound and one or more additional agents; as well as administration of the subject compound and one or more additional agent(s) in its own separate pharmaceutical dosage formulation. For example, a subject compound and an additional agent active with appetite suppressing activity (e.g., phentermine or topiramate) can be administered to the patient together in a single dosage composition such as a combined formulation, or each agent can be administered in a separate dosage formulation. Where separate dosage formulations are used, the subject compound and one or more additional agents can be administered concurrently, or at separately staggered times, e.g., sequentially. In some embodiments, the method further includes co-administering to the subject with the subject therapeutic agent, an antiepileptic agent. Antiepileptic agents of interest that find use in methods of co-administering include, but are not limited to, Acetazolamide, Carbamazepine, (Tegretol), Onfi (Clobazam), Clonazepam (Klonopin), Lamotrigine, Nitrazepam, Piracetam, Phenytoin, Retigabine, Stiripentol, Topiramate, and Carbatrol, Epitol, Equetro, Gabitril (tiagabine), Keppra (levetiracetam), Lamictal (lamotrigine), Lyrica (pregabalin), Gralise, Horizant, Neurontin, Gabarone (gabapentin), Dilantin, Prompt, Di-Phen, Epanutin, Phenytek (phenytoin), Topamax, Qudexy XR, Trokendi XR, Topiragen (topiramate), Trileptal, Oxtellar (oxcarbazepine), Depacon, Depakene, Depakote, Stavzor (valproate, valproic acid), Zonegran (zonisamide), Fycompa (perampanel), Aptiom (eslicarbazepine acetate), Vimpat (lacosamide), Sabril (vigabatrin), Banzel, Inovelon (rufinamide), Cerebyx (fosphenytoin), Zarontin (ethosuximide), Solfoton, Luminal (phenobarbital), Valium, Diastat (diazepam), Ativan (lorazepam), Lonopin, Klonopin (clonazepam), Frisium, Potiga (ezogabine), Felbatol (felbamate), Mysoline (primidone)

In some embodiments, the subject method is an in vitro method that includes contacting a sample with a subject compound. The protocols that can be employed in these methods are numerous, and include but are not limited to, serotonin release assays from neuronal cells, cell-free assays, binding assays (e.g., 5-HT2B receptor binding assays); cellular assays in which a cellular phenotype is measured, e.g., gene expression assays; and assays that involve a particular animal model for a condition of interest (e.g., Dravet syndrome, Lennox-Gastaut syndrome, Doose syndrome, West syndrome, and other refractory epilepsies) or symptoms or comorbidities associated with such conditions.

### Pharmaceutical Preparations

Also provided are pharmaceutical preparations. Pharmaceutical preparations are compositions that include a compound (either alone or in the presence of one or more additional active agents) present in a pharmaceutically acceptable vehicle. The term “pharmaceutically acceptable” means approved by a regulatory agency of the Federal or a state government or listed in the U.S. Pharmacopeia or other generally recognized pharmacopeia for use in mammals, such as humans. The term “vehicle” refers to a diluent, adjuvant, excipient, or carrier with which a compound of the invention is formulated for administration to a mammal.

The choice of excipient will be determined in part by the particular compound, as well as by the particular method used to administer the composition. Accordingly, there is a wide variety of suitable formulations of the pharmaceutical composition of the present invention.

The dosage form of a therapeutic agent employed in the methods of the present invention can be prepared by combining the therapeutic agent with one or more pharmaceutically acceptable diluents, carriers, adjuvants, and the like in a manner known to those skilled in the art of pharmaceutical formulation.

By way of illustration, the therapeutic agent can be admixed with conventional pharmaceutically acceptable carriers and excipients (i.e., vehicles) and used in the form of aqueous solutions, tablets, capsules, elixirs, suspensions, syrups, wafers, and the like. Such pharmaceutical compositions contain, in certain embodiments, from about 0.1% to about 90% by weight of the active compound, and more generally from about 1% to about 30% by weight of the active compound. The pharmaceutical compositions can contain common carriers and excipients, such as solubilizers, isotonic agents, suspending agents, emulsifying agents, stabilizers, preservatives, colorants, diluents, buffering agents, surfactants, moistening agents, flavoring agents and disintegrators, and including, but not limited to, corn starch, gelatin, lactose, dextrose, sucrose, microcrystalline cellulose, kaolin, mannitol, dicalcium phosphate, sodium chloride, alginic acid, vegetable or other similar oils, synthetic aliphatic acid glycerides, esters of higher aliphatic acids or propylene glycol, corn starch, potato starch, acacia, tragacanth, gelatin, glycerin, sorbitol, ethanol, polyethylene glycol, colloidal silicon dioxide, croscarmellose sodium, talc, magnesium stearate and stearic acid. Disintegrators commonly used in the formulations of this invention include croscarmellose, microcrystalline cellulose, corn starch, sodium starch glycolate and alginic acid. The compounds can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

In some embodiments, formulations suitable for oral administration can include (a) liquid solutions, such as an effective amount of the compound dissolved in diluents, such as water, or saline; (b) capsules, sachets or tablets, each containing a predetermined amount of the active ingredient, as solids or granules; (c) suspensions in an appropriate liquid; and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can include the active ingredient in a flavor, usually sucrose and acacia or tragacanth, as well as pastilles including the active ingredient in an inert base, such as gelatin and glycerin, or sucrose and acacia, emulsions, gels, and the like containing, in addition to the active ingredient, such excipients as are described herein.

In some cases, the compound is formulated for oral administration. In some cases, for an oral pharmaceutical formulation, suitable excipients include pharmaceutical grades of carriers such as mannitol, lactose, glucose, sucrose, starch, cellulose, gelatin, magnesium stearate, sodium saccharine, and/or magnesium carbonate. For use in oral liquid formulations, the composition can be prepared as a solution, suspension, emulsion, or syrup, being supplied either in solid or liquid form suitable for hydration in an aqueous carrier, such as, for example, aqueous saline, aqueous dextrose, glycerol, or ethanol, preferably water or normal saline. If desired, the composition can also contain minor amounts of non-toxic auxiliary substances such as wetting agents, emulsifying agents, or buffers.

Particular formulations of the invention are in a liquid form. The liquid can be a solution or suspension and can be an oral solution or syrup which is included in a bottle with a pipette which is graduated in terms of milligram amounts which will be obtained in a given volume of solution. The liquid solution makes it possible to adjust the solution for small children which can be administered anywhere from 0.5 mL to 15 mL and any amount between in half milligram increments and thus administered in 0.5, 1.0, 1.5, 2.0 mL, etc.

A liquid composition will generally consist of a suspension or solution of the compound or pharmaceutically acceptable salt in a suitable liquid carrier(s), for example, ethanol, glycerine, sorbitol, non-aqueous solvent such as polyethylene glycol, oils or water, with a suspending agent, preservative, surfactant, wetting agent, flavoring or coloring agent. Alternatively, a liquid formulation can be prepared from a powder for reconstitution.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Binding of Fenfluramine and Norfenfluramine to 47 Candidate Receptors

The importance of 5-HT receptor subtypes in mediating fenfluramine's anti-seizure activity has been reported in the literature. See Dinday and Baraban, Large-scale phenotype-based antiepileptic drug screening in a zebrafish model of Dravet syndrome, eNeuro 2(4), pp 1-19, July/August 2015. To further elucidate the mechanism of action underlying fenfluramine's efficacy in controlling seizures, the binding potency of fenfluramine and norfenfluramine at other receptors previously identified in the literature as being linked to epilepsy was determined.

A list of 47 candidate receptors were identified by a literature search for receptors reported as being implicated in seizure activity. The inhibition ratios of test articles on binding of tracer to each of the 47 candidate receptors were then calculated to assess binding potency of racemic fenfluramine and norfenfluramine with respect to each of the candidate receptors.

### Example 1

### Materials and Methods

### Identification of Candidate Receptors

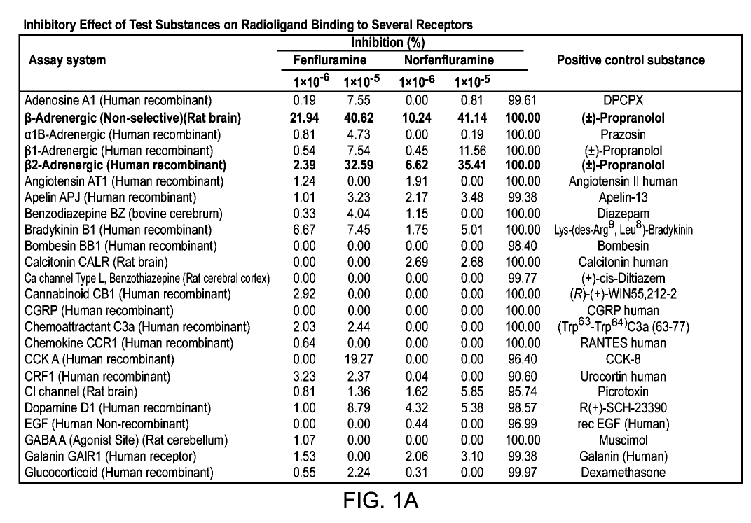

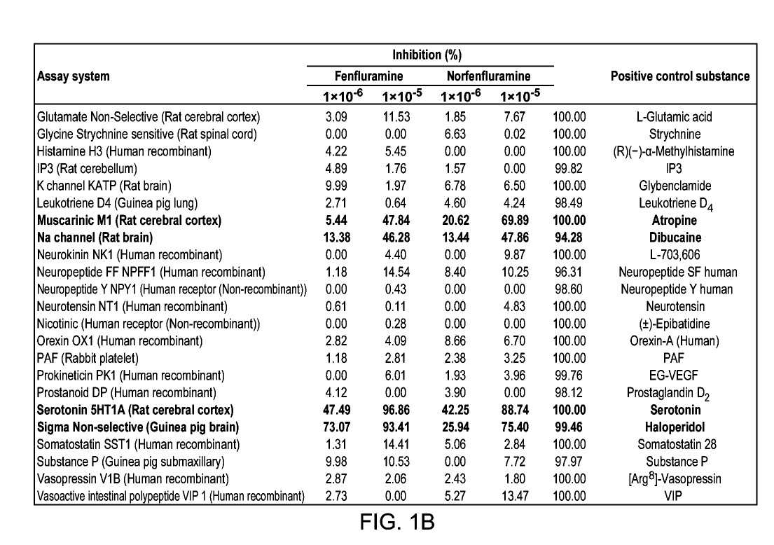

A set of 47 candidate receptors (see FIG. 1A and FIG. 1B) reported to be implicated in epileptic seizure activity was identified from a comprehensive literature search. A competitive binding assay was used to assess binding for each of the 47 receptors by calculating the inhibition ratios of racemic mixtures of fenfluramine and norfenfluramine, respectively, on the binding of tracer to various receptors using a competitive radioligand binding assay.

### Reagents

Test Articles: Fenfluramine and norfenfluramine were obtained from Zogenix and stored under protection from light. Test articles were then weighed and dissolved in DMSO to prepare test article solutions at 100-fold higher concentrations of the final concentrations used in the assays shown below, then diluted 10-fold with Milli-Q water (tap water purified with an ultrapure water purifier) just before use.

Positive Controls: Similarly, positive control substances were weighed, dissolved in DMSO, and diluted serially with DMSO to prepare the solutions at 100-fold higher concentrations of the final concentrations shown below, then diluted 10-fold with Milli-Q water just before use.

Other Assay Reagents: Other reagents were obtained from readily available commercial sources. All reagents were of the guaranteed grade or equivalents. Milli-Q water was used.

### Assay Protocol

Test article solutions were prepared as described in the Materials and Methods section above. The prepared solutions were then diluted 10-fold with Milli-Q water to prepare test article solutions at final concentrations of 1×10⁻⁶ and 1×10⁻⁵ mol/L. Positive control solutions were prepared as described in the Materials and Methods section above, then diluted 10-fold with Milli-Q water to prepare positive control substance solutions just before use to final concentrations of 1×10⁻⁶ or 1×10⁻⁵ mol/L.

Duplicate samples of the assay solutions were assayed once.

### Example 1

### Data Analysis, Acceptance Criteria, & Data Processing

Inhibition Ratios, IC₅₀ values, and K_{D} values were determined for each of the 47 candidate receptors

Inhibition Ratios: Inhibition ratios were calculated as follows:

Inhibition ratios (%)=100−binding ratio

Binding ratio=[(B−N)/(B0−N)]×100 (%), where

B is Bound radioactivity in the presence of the test article (individual value)

B0 is Total bound radioactivity in the absence of the test article (mean value); and

N is Non-specific bound radioactivity (mean value).

When the inhibition ratio was less than 0% or over 100%, it was calculated as 0% or 100%, respectively. The inhibition ratios of the positive control substances were calculated in the same way as those of the test articles. Microsoft® Excel 2007 (Microsoft Corporation) was used for the data processing.

The acceptance criterion of assay values was an inhibition ratio of the positive control substance of 80% or more. Furthermore, the acceptance criterion of assay values was that the inhibition ratios from duplicate assay values of the test articles and positive control substances were within 10% of the mean of the inhibition ratios. Since all the assay values met the above criteria, re-assay was not performed.

IC₅₀ values: IC₅₀ values were determined as follows. The mean inhibition ratio of the test articles and positive control substances calculated from duplicate samples were expressed as % and rounded off at the third decimal place to two decimal places. The ratio ((B−N)/(B₀−N)) of specific bound radioactivity in the presence of the test substance (B−N) to total bound radioactivity in the absence of the test substance (B₀−N) was transformed by the logit transformation and plotted to the final concentrations of the test substance on a logarithmic scale (Scatchard plot). The concentration-response curve was regressed to the following logit-log expression:

Y=aX+b

{Y=logit y=ln [y/(1−y)], where y=(B−N)/(B₀−N)}, where

X=log x, where x is the final concentrations of the test substances), and

(a, b=constant)

Microsoft® Excel 2007 (Microsoft Corporation) was used for data processing.

IC₅₀ values were then calculated from the regression equations. When the mean inhibition ratio of the test article was out of the range from 5% to 95%, this value was excluded, and the IC₅₀ value was calculated using the values within the acceptable range. When the value from one of triplicate samples was below zero or exceeds 100%, the mean inhibition ratio of the concentration was used for calculation of IC₅₀ values.

The inhibition ratios of the test substances to each concentration were expressed with mean values of triplicate samples in a unit of %. The values were rounded off at the third decimal place and expressed to two decimal places. The IC₅₀ value was expressed with index number in a unit of mol/L. The values were rounded off at the third decimal place and expressed to two decimal places (data not shown).

K_{D} values: K_{D} values for fenfluramine, norfenfluramine, and their enantiomers were determined by Scatchard Analysis (n=2). Radioactivity was converted to the concentration of the tracer. B/F and B were plotted on vertical axis and horizontal axis, respectively, and the linear regression was achieved. The K_{d} and Bₘₐₓ values were calculated using the following equation. Microsoft® Excel 2007 was used for data processing.

B/F=−1/Kd×(B−Bmax), where

B=Concentration of bound radioactivity (mean value),

F=Concentration of unbound radioactivity (mean value).

−1/Kd:=Slope, and

Bₘₐₓ: Intercept of B

Ki values: Ki values were calculated from IC₅₀ values and Kd values using the following equations:

Ki=IC₅₀/(1+L/Kd)

L is the concentration of bound ligand.

Data Processing: Microsoft® Excel 2007 (Microsoft Corporation) was used for data processing.

### Example 1

### Results and Conclusion

Results are presented in tabular form. See FIG. 1A and FIG. 1B.

Based on the results of the competitive binding assays, fenfluramine and norfenfluramine were found to significantly inhibit receptor binding of positive controls by the following receptors: β-Adrenergic (Non-selective) (Rat brain), β2-Adrenergic (Human recombinant), Muscarinic M1 (Rat cerebral cortex), Na channel (Rat brain), serotonin 5-HT1A (rat cerebral cortex) and Sigma non-selective (Guinea pig brain)

### Example 2

### Determination of IC₅₀, Kd and Ki for Fenfluramine and Norfenfluramine Binding to Selected Receptors

IC₅₀, Kd and Ki values of fenfluramine and norfenfluramine were determined for the following receptors: β-Adrenergic (Non-selective) (Rat brain), β2-Adrenergic (Human recombinant), Muscarinic M1 (Rat cerebral cortex), Na channel (Rat brain), serotonin 5-HT1A (rat cerebral cortex) and Sigma non-selective (Guinea pig brain).

### Example 2

### Materials and Methods

### Preparation of Reagents

The test articles were obtained from Zogenix Inc. and stored as described in the Materials and Methods section of Example 1 above.

### Assay Protocols and Sample Replication

The binding assays for the receptors were repeated as described in the Materials and Methods section of Example 1 above using the specified range of concentrations. Triplicate samples of the solutions were assayed once.

Seven test concentrations of both reagents were used for each receptor assay. For the B-adrenergic, B2-adrenergic, muscarinic M1 and Na channel assays, test article concentrations of 1×10⁻⁷, 3×10⁻⁷, 1×10⁻⁶, 3×10⁻⁶, 1×10⁻⁵, 3×10⁻⁵, and 1×10⁻⁴ mol/L were used. For the serotonin 5-HT1A and sigma receptors, 1×10⁻⁸, 3×10⁻⁸, 1×10⁻⁷, 3×10⁻⁷, 1×10⁻⁶, 3×10⁻⁶, and 1×10⁻⁵ mol/L were used.

Positive control substances were prepared at 100× concentrations, as described in the Materials and Methods section above. Seven concentrations were used for each assay. For β-adrenergic, β2-adrenergic, muscarinic M1, serotonin 5-HT1A, and sigma, concentrations of 1×10−10, 3×10−10, 1×10−9, 3×10−9, 1×10−8, 3×10−8, and 1×10−7 were used. For the Na channel assay, concentrations of 1×10−8, 3×10−8, 1×10−7, 3×10−7, 1×10−6, 3×10−6, and 1×10−5 mol/L were used.

### Data Analysis

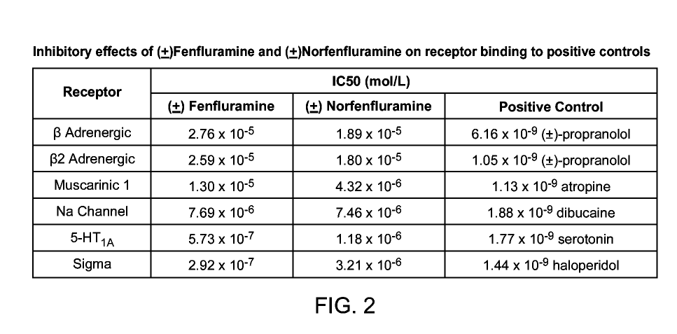

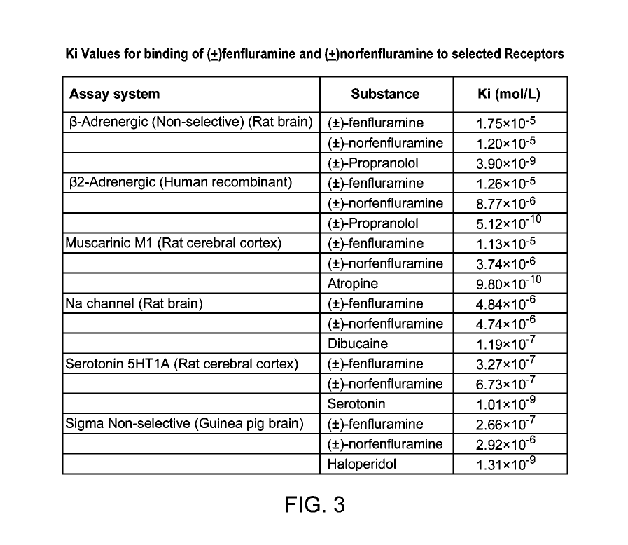

Inhibition ratios, IC₅₀, Kd and Ki values for racemic fenfluramine, racemic norfenfluramine and positive control substances were calculated as described above. IC₅₀ values calculated for racemic fenfluramine, norfenfluramine, and known positive controls for each of the receptors tested are shown in FIG. 2; corresponding Kᵢ values are shown in FIG. 3.

### Example 2

### Results and Conclusion

These results show that racemic fenfluramine and racemic norfenfluramine show moderate binding of the β-1 adrenergic, β2 adrenergic, muscarinic M1, Na channel, 5-HT1A, and sigma receptors relative to positive controls.

### Example 3

### Determination of IC₅₀, Ki and Kd Values for Binding of Enantiomers of Fenfluramine and Norfenfluramine to Selected Receptors

The therapeutic effects of some pharmaceutical agents, notably citalopram, are associated with one stereoisomer while unwanted side effects are associated with the other, thus in some cases it is possible to obtain therapeutic benefits while minimizing side effects by administering a pure enantiomer of a chiral therapeutic agent.

Most of fenfluramine's undesired side effects are attributed to the effects of its metabolite norfenfluramine, particularly at the 5-HT2B receptor. Therefore, as a first step towards determining whether the enantiomers of fenfluramine and/or norfenfluramine had disparate effects as compared to racemic mixes of those compounds, the binding potency (% inhibition), IC₅₀, Ki, and Kd values for the β adrenergic, β2 adrenergic, muscarinic M1, Na channel, 5-HT1A, 5-HT2A, 5-HT2B, 5-HT2C, 5-HT5A, 5-HT7, sigma-1, and sigma-1 receptors for all six compounds were determined, and values for the racemic mixes and both enantiomers compared.

### Example 3

### Materials and Methods

### Reagents

Test articles were obtained and stored as follows:

TABLE 3
Sources of Test Materials

| | Compound | | | |
|---|---|---|---|---|
| | (+)-Fenflur- | (−)-Fenflur- | (+)-Norfen- | (−)-Norfen- |
| | amine | amine | fluramine | fluramine |
| Lot No. | SLBF9148V | 059H0827 | 059H0827 | IB53B |
| Formula | 267.72 | 267.72 | 239.67 | 239.67 |
| Wt. | | | | |
| Receipt | 25 mg | 25 mg | 20 mg | 20 mg |
| Amount | | | | |
| Storage | Room | Room | Refrigerated | Refrigerated |
| Conditions | temperature, | temperature, | (set at 4° C.), | (set at 4° C.), |
| | protected | protected | Protected | Protected |
| | from light | from light | from light | from light |

Test article solutions were prepared at 100× of final as described in the Materials and Methods section of Example 1 above, then diluted 10× just prior to use.

Test article solutions were prepared at 100× concentrations, as described in the Materials and Methods section above. Seven test concentrations of both reagents were used for each receptor assay. For the B-adrenergic, B2-adrenergic, muscarinic M1, Na channel, serotonin 5-HT2A, serotonin 5-HT2B, and serotonin 5-HT7 assays, test article concentrations of 1×10⁻⁷, 3×10⁻⁷, 1×10⁻⁶, 3×10⁻⁶, 1×10⁻⁵, 3×10⁻⁵, and 1×10⁻⁴ mol/L were used. For the serotonin 5-HT1A, 5-HT2C, Sigma 1, and Sigma 2 receptors, 3×10⁻⁸, 1×10⁻⁷, 3×10⁻⁷, 1×10⁻⁶, 3×10⁻⁶, 1×10⁻⁵, and 3×10⁻⁵ mol/L, were used.

Other reagents were obtained from readily available commercial sources.

The positive control substances were prepared at 100× concentrations, as described in the Materials and Methods section above. Seven concentrations were used for each assay. For β-adrenergic, β2-adrenergic, muscarinic M1, serotonin 5-HT1A, and sigma, concentrations of 1×10⁻¹⁰, 3×10⁻¹⁰, 1×10⁻⁹, 3×10⁻⁹, 1×10⁻⁸, 3×10⁻⁸, and 1×10⁻⁷ were used. For the Na channel assay, concentrations of 1×10⁻⁸, 3×10⁻⁸, 1×10⁻⁷, 3×10⁻⁷, 1×10⁻⁶, 3×10⁻⁶, and 1×10⁻⁵ mol/L were used.

### Assay Protocols

Radioligand binding assays and described in Example 1 above were repeated using racemic mixes and stereoisomers of fenfluramine and norfenfluramine for the following receptors: β-Adrenergic (Non-selective) (Rat brain), β2-Adrenergic (Human recombinant), Muscarinic M1 (Rat cerebral cortex), Na channel (Rat brain), serotonin 5-HT1A (rat cerebral cortex), Serotonin 5-HT1A (Rat cerebral cortex), Serotonin 5-HT2A (Human recombinant), Serotonin 5-HT2B (Human recombinant) Serotonin 5-HT2C (Human recombinant), Serotonin 5-HT7 (Human recombinant), Sigma non-selective (Guinea pig brain), Sigma 1 (Guinea pig brain), and Sigma 2 (Guinea pig brain).

Triplicate samples of the assay solutions were assayed once.

### Example 3

### Data Analysis and Results

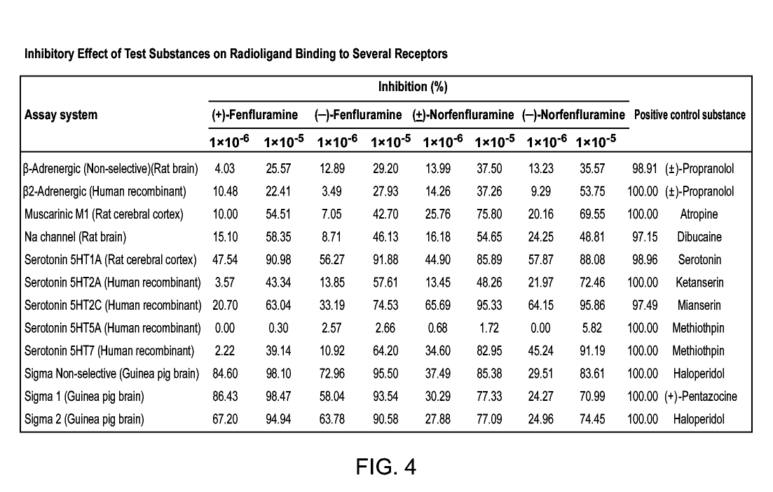

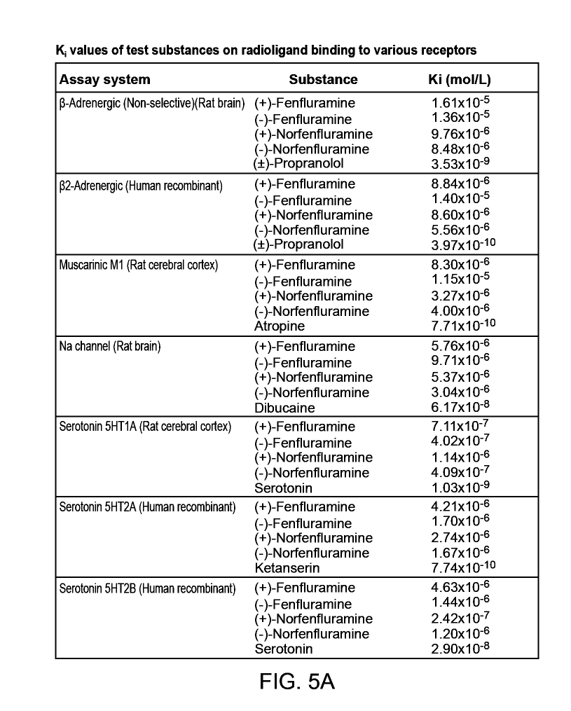

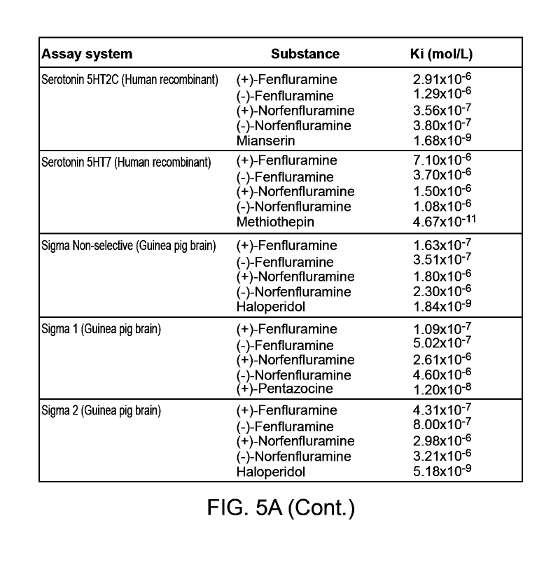

Ki values were calculated for (+) and (−) fenfluramine and for (+) and (−) norfenfluramine for the following receptors using competitive inhibition assays: Beta-adrenergic. Beta2-adrenergic, Muscarinic M1, Na Channel, Sigma (nonselective), Sigma 1, and Sigma 2. % Inhibition, IC₅₀, Kd, and Ki values were determined as above. Results are shown in FIG. 4, FIG. 5A and FIG. 5B.

For 5-HT1A, there was no difference in binding of the fenfluramine enantiomers. (−)norfenfluramine showed slightly tighter binding to the receptor than (+)norfenfluramine (Ki=4.09×10⁻⁷ and 1.14×10⁻⁶).

For 5-HT2A, 5-HT2C or 5-HT7, there were no differences between binding of the test compounds and their enantiomers (data not shown).

For 5-HT2B, there was no difference in binding of the fenfluramine enantiomers. (+)norfenfluramine showed slightly tighter binding to the receptor than (−)norfenfluramine (Ki=2.42×10⁻⁷ and 1.20×10⁻⁶ respectively (data not shown)

For the beta-adrenergic receptor, the Na channel, and the sigma receptors, there were no differences in the Ki values of any of the test compounds.

For the beta2 adrenergic receptor, (+)fenfluramine showed slightly tighter binding to the receptor than (−) fenfluramine (Ki=8.84×10−6 and 1.40×10−5 respectively). There was no difference in binding of the enantiomers of norfenfluramine.

For the muscarinic M1 receptor, (+)fenfluramine showed slightly tighter binding to the receptor than (−)fenfluramine (Ki=8.30×10−6 and 1.15×10−5 respectively). There was no difference in binding of the enantiomers of norfenfluramine.

These results demonstrate that, for the receptors tested, there is little or no difference in binding activity between the enantiomers of fenfluramine and little or no difference in binding activity between the enantiomers of norfenfluramine.

### Example 4

### Functional Assays of Fenfluramine and Norfenfluramine and Their Enantiomers for Activity at Selected Receptors

The effects of fenfluramine and norfenfluramine, and their enantiomers (collectively, “test compounds”) on the activity of selected receptors were assessed using cell- and tissue-function assays. The activity of the test compounds at the β adrenergic, β2 adrenergic, and β3 adrenergic receptors were assessed using cell-based GPCR assays. Activity at the Muscarinic M1 receptor was assessed by measuring their effects on Ca2+ ion mobilization using a fluorometric detection method. Activity for the 5-HT1A receptor was determined by measuring their effects on impedance modulation using a CellKey (CDS) detection method. Cellular agonist effect was calculated as a % of control response to a known reference agonist for each target and cellular antagonist effects was calculated as a % inhibition of control reference agonist response for each target. EC50 and IC₅₀ values were also determined.

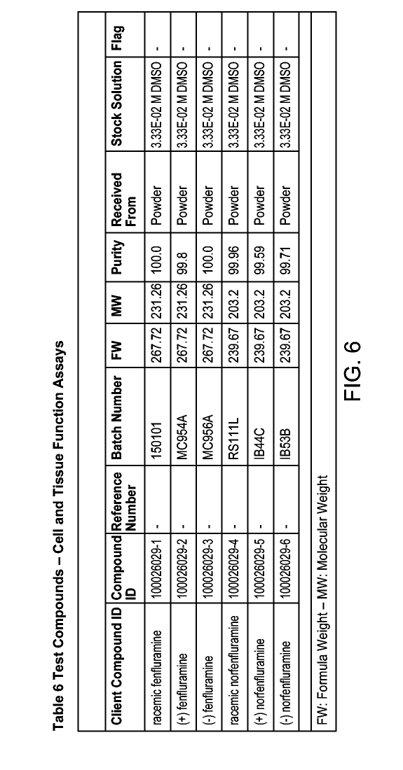

Samples of fenfluramine and norfenfluramine racemates and enantiomers were obtained from Zogenix. 3.33e-2 stock solutions of each test compound in DMSO were prepared and stored See FIG. 6.

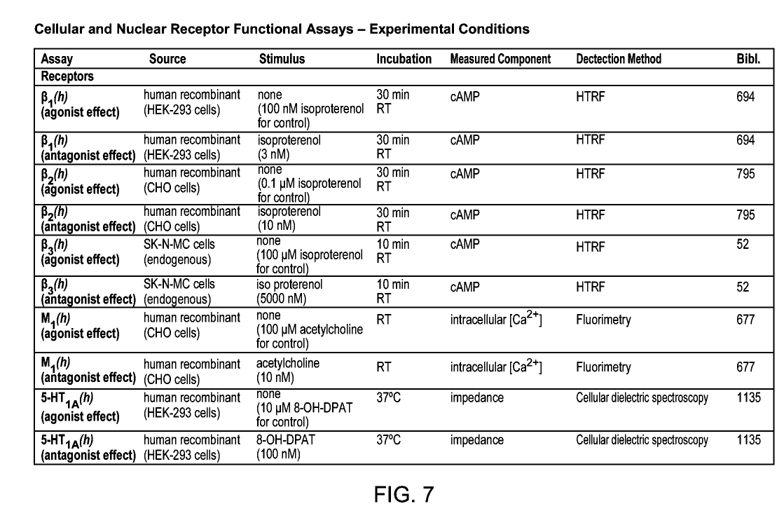

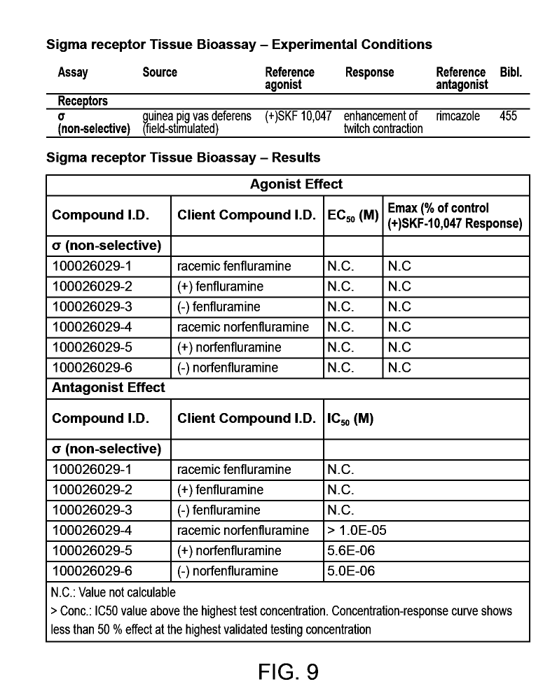

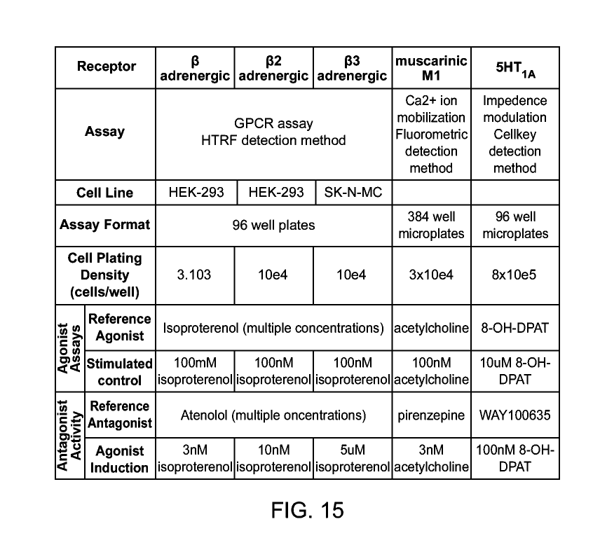

Experimental conditions for cell function assays are shown in FIG. 7. Experimental conditions for the sigma receptor tissue activity appear in FIG. 9A. See FIG. 15 for cell plating densities, reference agonists, and reference antagonists.

### 4(A)

### Adrenergic Receptors

The effects of racemic fenfluramine and norfenfluramine as well as their enantiomers (collectively, “test compounds”) on the activity of the β-1 adrenergic, β2 adrenergic, and β3 adrenergic receptors (“beta adrenergic receptors”) using cell-based GPCR assays.

### Adrenergic Receptors—Materials and Methods

### Adrenergic Receptors—Cells

Transfected HEK-293 cells expressing human β-1 adrenergic receptor, human β-2 adrenergic receptor, and human β3 adrenergic receptor, respectively, were prepared using cloned human cDNA. See H FRIELLE, T., COLLINS, S., DANIEL, K. W., CARON, M. G., LEFKOWITZ, R. J., KOBILKA, B. K. (1987), Cloning of the cDNA for the human beta1-adrenergic receptor, Proc. Natl. Acad. Sci. U.S.A., 84,7920, and BAKER, J. G. (2005) The selectivity of Beta-adrenoreceptor antagonists at the human Beta1, Beta2 and Beta3 adrenoreceptor, Brit. J. Pharmacol., 144: 317).

Human SK-N-MC cells expressing endogenous β3 adrenergic receptor were obtained from a commercial source.

Transfected cells were suspended in HBSS buffer (Invitrogen) complemented with 20 mM HEPES (pH 7.4) and 500 μM IBMX. The suspension buffer for the β3-adrenergic receptor assays additionally contained 1 uM propranolol. The cells were then distributed in 96 well microplates (see FIG. 15 for plating densities).

### Adrenergic Receptors—Agonist Activity Assay

Agonist activity of the test compounds at the β adrenergic, β2 adrenergic, and β3 adrenergic receptors, respectively, was assessed by measuring their effects on cAMP production in transfected cells expressing each of the receptors using the HTRF detection method.

After cells were plated, HBSS (basal control), the test compounds (test wells), and reference agonist (stimulated control wells and reference wells) were then added. Additionally, the reference agonist was also added to stimulated control wells. All wells contained a final reaction volume of 20 uL. Test compounds were added by first preparing 100× concentrated solutions in solvent, then diluting to 10× concentration solution in HBSS and 0.1% BSA just prior to use. DMSO concentration did not exceed 1%. The microplates were then incubated for 30 min at room temperature.

Following incubation, the cells were lysed and both a fluorescence acceptor (D2-labeled cAMP) and fluorescence donor (anti-cAMP antibody labeled with europium cryptate) were added. After 60 min at room temperature, the fluorescence transfer was measured at λex=337 nm and λem=620 and 665 nm using a microplate reader (Rubystar, BMG).

The cAMP concentration was determined by dividing the signal measured at 665 nm by that measured at 620 nm (ratio). The results are expressed as a percent of the control response determined for the stimulated control wells. In addition to the stimulated control wells, the standard reference agonist (isoproterenol) was tested in each experiment at several concentrations to generate a concentration-response curve from which its EC50 value is calculated.

### Adrenergic Receptors—Antagonist Activity Assay

Antagonist activity of the test compounds at the β adrenergic, β2 adrenergic, and β3 adrenergic receptors, respectively, was assessed by measuring their effects on agonist-induced cAMP production in transfected cells expressing each of the receptors using the HTRF detection method.

After plating, the cells were induced by adding reference agonist. See FIG. 15 for reference agonists and concentrations used for each assay. For basal control measurements, separate assay wells did not contain isoproterenol. The cells were then incubated 30 minutes at room temperature.

Subsequently, the cells were lysed and a fluorescence acceptor (D2-labeled cAMP) and a fluorescence donor (anti-cAMP antibody labeled with europium cryptate) were added to the wells. After 60 min at room temperature, the fluorescence transfer was measured at λex=337 nm and λem=620 and 665 nm using a microplate reader (Rubystar, BMG).

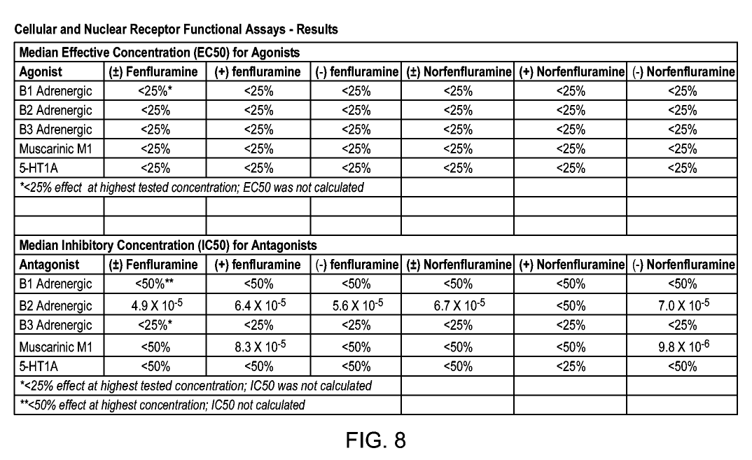

cAMP concentration was then determined by dividing the signal measured at 665 nm by that measured at 620 nm (ratio). The results are expressed as a percent inhibition of the control response to 3 nM isoproterenol. See FIG. 8.

Standard reference antagonists were tested in each experiment at several concentrations to generate a concentration-response curve from which its IC₅₀ value is calculated.

### Adrenergic Receptors—Data Analysis and Results

Results are expressed as a percent of control agonist response and as a percent inhibition of control agonist response obtained in the presence of the test compound:

(measured response/control response)×100,

100−[(measured response/control response)*100)

EC₅₀ values (concentration producing a half-maximal response) and IC₅₀ values (concentration causing a half-maximal inhibition of the control agonist response) were determined by non-linear regression analysis of the concentration-response curves generated with mean replicate values using Hill equation curve fitting:

Y=D+{(A−Z/[1+(C/C₅₀)^{nH}]}, where

Y=response,

A=left asymptote of the curve,

D=right asymptote of the curve,

C=compound concentration, and

C₅₀=EC₅₀ or IC₅₀, and nH=slope factor.

The analysis was performed using software developed at Cerep (Hill software) and validated by comparison with data generated by the commercial software SigmaPlot® 4.0 for Windows® (© 1997 by SPSS Inc.).

For the antagonists, the apparent dissociation constants (K_{B}) were calculated using the modified Cheng Prusoff equation:

K_{B}=IC₅₀/[1+(A/EC_{50A})], where

A=concentration of reference agonist in the assay, and

EC_{50A}=EC₅₀ value of the reference agonist.

Results showing an inhibition or stimulation higher than 50% are considered to represent significant effects of the test compounds. Results showing a stimulation or an inhibition lower than 25% are not considered significant and mostly attributable to variability of the signal around the control level.

Summary results of the beta-adrenergic functional assays appear in FIG. 8.

### Adrenergic Receptors—Conclusions

The results of the GPCR assays support the conclusion that none of the test compounds have agonist activity at any the B1-adrenergic, B2-adenergic, or B3 adrenergic receptor.

Further, these results support the conclusion that (±)fenfluramine, both fenfluramine enantiomers, (±)norfenfluramine and (−)norfenfluramine all have antagonist activity at the beta-2 adrenergic receptor, while (+)norfenfluramine has no effect on that receptor.

### 4(B)

### Muscarinic M1 Receptor

The activity of the test compounds at the muscarinic M1 receptor was assessed by measuring their effects on Ca2+ ion mobilization in transfected CHO cells expressing the receptor using a fluorometric detection method.

### Muscarinic M1 Receptor—Materials and Methods

### Muscarinic M1 Receptor—Cells

Human muscarinic M1 receptor cDNA was cloned and used to transfect CHO cells. See SUR, C., MALLORGA, P. J., WITTMANN, M., JACOBSON, M. A., PASCARELLA, D., WILLIAMS, J. B., BRANDISH, P. E., PETTIBONE, D. J., SCOLNICK, E. M. and CONN, P. J. (2003), N-desmethylclozapine, an allosteric agonist at muscarinic 1 receptor, potentiates N-methyl-D-aspartate receptor activity. Proc. Natl. Acad. Sci. U.S.A., 100: 13674.

Transfected CHO cells were suspended in DMEM buffer (Invitrogen) complemented with 0.1% FCSd, then distributed in 384 well microplates at a density of 3×104 cells/well.

### Muscarinic M1 Receptor—Agonist Activity Assay

Agonist activity of the test compounds at the Muscarinic M1 receptor was assessed by measuring their effects on changes in Ca2+ ion mobilization in transfected CHO cells expressing the receptor using a fluorometric detection method.

After plating, a fluorescent probe (Fluo4 direct, Invitrogen), mixed with probenicid in HBSS buffer (Invitrogen) complemented with 20 mM Hepes (Invitrogen) (pH 7.4), was added into each microplate well and equilibrated with the cells for 60 min at 37° C. then 15 min at 22° C.

Thereafter, the assay plates were positioned in a microplate reader (CellLux, PerkinElmer). HBSS buffer, test compounds, and reference agonist were then added to basal control and test, and reference wells, to a final reaction volume of 90 uL. Test compounds were added by first preparing 333× concentrated stock solutions in DMSO, then diluted to [10×] in HBSS and 0.1% BSA just prior to use. The maximum tolerable DMSO concentration was 0.3%. Separate stimulated control wells contained acetylcholine at 100 nM.

Reference wells containing varying concentrations of the reference agonist acetylcholine were included in each experiment. The resulting data was plotted to generate a concentration-response curve from which its EC50 value was calculated.

Changes in fluorescence intensity, which vary proportionally to the free cytosolic Ca2+ ion concentration, were then measured. The results are expressed as a percent of the control response to 100 nM acetylcholine.

### Muscarinic M1 Receptor—Antagonist Activity Assay

Antagonist activity of the test compounds at the Muscarinic M1 receptor was assessed by measuring their effects on agonist-induced cytosolic Ca2+ ion mobilization in transfected CHO cells expressing the receptor using a fluorometric detection method.

After plating, a fluorescent probe (Fluo4 NW, Invitrogen), mixed with probenicid in HBSS buffer (Invitrogen) complemented with 20 mM Hepes (Invitrogen) (pH 7.4), was added into each well and equilibrated with the cells for 60 min at 37° C., followed by a second incubation for 15 min at 22° C.

Thereafter, the assay plates were positioned in a microplate reader (CellLux, PerkinElmer). After a 5-min incubation, 3 nM acetylcholine was added to all except the basal control wells to a total reaction volume of 100 uL, and changes in fluorescence intensity which vary proportionally to the free cytosolic Ca2+ ion concentration, were measured.

Test compounds were added by first preparing [333×] stock solutions of each compound in solvent. The stock solutions were then diluted to [10×] in HBSS and 0.1% BSA just prior to use. Maximum tolerable DMSO concentration was 0.3%.

The standard reference antagonist pirenzepine was tested in each experiment at several concentrations to generate a concentration-response curve from which its IC₅₀ value was calculated.

### Muscarinic M1 Receptor Activity—Results, Data Analysis, and Conclusion

Results are shown in FIG. 8. Data analysis was as in Example 4(A) above.

These results support the conclusion that (+)fenfluramine has antagonist activity at the muscarinic M1 receptor, while the remaining test compounds have no significant effects.

### 4(C)

### Serotonin 5-HT1A Receptor

The activity of the test compounds at the 5-HT1A receptor was determined by monitoring their effects on impedance modulation in transfected HEK293 cells expressing the receptor using a CellKey (CDSD) detection method.

### 5-HT1A Receptor—Materials and Methods

### 5-HT1A Receptor—Transfected Cells

Human serotonin 5-HT1A receptor cDNA was cloned and used to transfect HDK-293 cells. MARTEL, J-C., ASSIE, M-B., BARTIN, L., DEPOORTERE, R., CUSSAC, D. and NEWMAN-TANCREDI, A. (2009), 5-HT1A receptors are involved in the effects of xaliprofen on G-protein activation, neurotransmitter release and nociception, Brit J Pharmacol, 158: 232.

Cells were suspended in HBSS buffer (Invitrogen) complemented with 20 mM HEPES (pH 7.4) and 0.1% BSA, then seeded onto 96-well plates coated with fibronectin at 8×105 cells/well and allowed to equilibrate for 60 min at 37° C.

### 5-HT1A Receptor—Agonist Activity Assay

Agonist activity of the test compounds at the 5-HT1A receptor was assessed by measuring their impedance modulation effects on transfected HEK293 cells expressing the receptor using the CellKey cellular dielectric spectroscopy (CDS) detection method.

Following cell seeding and equilibration, the microplates were placed onto the CellKey system. Then HBSS (basal control wells), 10 uM 8-OH-DPAT (reference wells and stimulated control wells), and the test compounds (test wells) were added. Test compounds were added by first preparing 1000× stock solutions in solvent, then diluting to 10× of final reaction volume in 10× HBSS and 0.1% BSA. The maximum tolerable DMSO concentration was 0.1%. Reference wells contained various concentrations of the standard reference agonist 8-OH-DPAT.

All solutions were added simultaneously to all 96 wells using an integrated fluidics system to a final reaction volume of 150 uL

Finally, impedance measurements were monitored for 20 minutes after ligand addition at a temperature of 37 C.

Data from the reference wells were plotted to generate a concentration-response curve which was then used to calculate EC50 values for the test compounds.

### HT-1A Receptor—Antagonist Activity

Antagonist activity of the test compounds at the 5-HT1A receptor was assessed by measuring their effects on agonist-induced impedance modulation in transfected HEK-293 cells expressing the receptor using the CellKey (CDS) detection method.

Following cell seeding and equilibration, the microplates were placed onto the CellKey system. Then HBSS (basal control wells and stimulated control wells), and the test compounds (test wells) were added. Test compounds were added by first preparing 1000× stock solutions in solvent, then diluting to 10× of final reaction volume in 10× HBSS and 0.1% BSA. The maximum tolerable DMSO concentration was 0.1%. Additionally, reference wells containing various concentrations of the standard reference antagonist WAY100634 were prepared for each experiment.

The plates were then preincubated for 25 minutes at 37 C.

After the preincubation, HBSS (basal control wells) and 100 nM 8-OH-DPAT (stimulated control wells) were added.

All solutions were added simultaneously to all 96 wells using an integrated fluidics system. The final reaction volume was 167 uL

Finally, impedance measurements are monitored for 20 minutes at a temperature of 37 C.

Data from the reference wells were plotted to generate a concentration-response curve which was then used to calculate IC₅₀ values for the test compounds.

### 5-HT1A Receptor—Results, Data Analysis, and Conclusion

Data analysis was as in 4(A) above. Results are shown in FIG. 8.

These results support the conclusion that none of the test compounds have either agonist or antagonist activity at the 5-HT1A receptor.

### Example 5

### Ion Channel Profiling

Based on the binding study results obtained for sodium channel (see Example 1 and Example 2, infra), electrophysiologic (“patch clam”) assays were conducted to assess the activity of the test compounds on the following ion channel targets: hNav1.1, hNav1.2, hNav1.3, hNav1.4, hNav1.5, hNav1.6, hNav1.7, and hNav1.8.

### Ion Channel Target Profiling—Material & Methods

Electrophysiological assays were conducted to profile racemic fenfluramine and pure stereoisomers of both compounds for activities on 8 sodium ion channel targets specified above using the IonFlux HT automated patch clamp system.

All compounds were assayed as five (5) point concentration responses, and IC₅₀ values were estimated based on results. Assays were conducted by Eurofins's IonChannelProfiler™ services using their proprietary PRECISION ion channel stable cell lines and the IONFLUX HT patch clamp system (Eurofins Pharma Bioanalytics Services US Inc., St. Charles, Mo., USA).

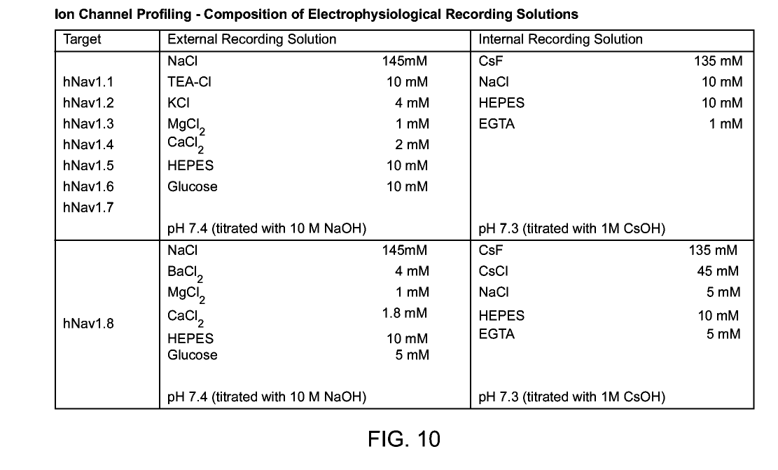

Reaction condition and recording solution compositions are shown in FIG. 10. Test compounds were supplied by Zogenix. All other reagents were of the guaranteed grade or equivalents and were obtained from commercial sources. Milli-Q water was used.

Test compound(s) were prepared in DMSO to concentrations that were 300× the final top assay concentration(s). All test compounds were tested at concentrations of 0.37, 1.11, 3.33, 10, and 30 μM. 0.33% DMSO was used as a vehicle control for all assays.

Positive controls were as follows. For hNav1.1: tetracain at 4.1×10⁻¹ μM, 1.23 μM, 3.7 μM, 11.1 μM, 33.33 μM, and 100 μM. For hNav1.2, hNav1.3, hNav1.5, hNav1.6. Nav1.7: lidocaine at 6.86 μM, 20.58 μM, 61.73 μM, 185.19 μM, 555.56 μM, 1,666.67 μM, and 5,000 μM. For hNav1.4, lidocaine at 20.58 μM, 61.73 μM, 185.19 μM, 555.56 μM, 1,666.67 μM, and 5,000 μM. For hNav1.8: AB03467 at 1×10⁻⁵ μM, 1×10⁻⁶ μM, 1×10⁻⁷ μM, 1×10⁻⁸ μM, 1×10⁹ μM, 1×10⁻¹⁰ μM, and 1×10⁻¹¹ μM.

On the day of the assay, dose-responses were prepared by 3-fold serial dilution in DMSO from the top concentration and aliquots were taken out from the respective concentrations and adding appropriate amounts of external buffer. All wells included a final DMSO concentration of 0.33% including all control wells.

Increase in drug-induced block of voltage-activated sodium channels (hNav1.1 to hNav1.8) upon application of a train of pulses, with the requirement for an incomplete block during the first pulse and incomplete recovery during the interval between pulses. An example is Tetracaine and lidocaine inhibition, which show much stronger inhibition at pulse 20 than at pulse 1.

Pulse Protocols for each of the Nav subunits tested appear below.

IonFlux HT 20-Pulse Protocol—hNav1.1 to hNav1.7

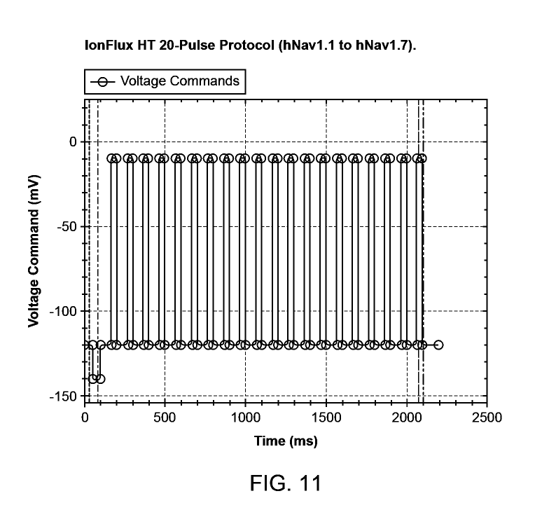

A schematic of the pulse protocol used is shown in FIG. 11. Cells were held at −120 mV for 50 ms before stepping to −10 mV for 10 ms to activate Nav1.1 to Nav1.7 currents and stepped back to −120 mV for 90 ms (to completely recover from inactivation, however channels that had drugs bound to them will not recover from inactivation) and this pattern was repeated 20 times with a sweep interval of 100 ms (10 Hz). Each concentration of compound was applied for 2 minutes. The Nav1.1 to Nav1.7 experiments were performed at room temperature (approximately 22° C.).

IonFlux HT 20-Pulse Protocol—hNav1.8

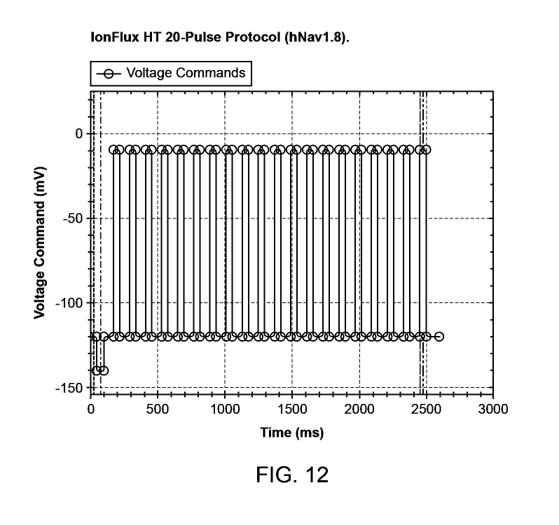

A schematic is shown in FIG. 12. Cells were held at −120 mV for 50 ms before stepping to −10 mV for 50 ms to completely inactivate the hNav1.8 channels (pulse 1), and stepped back to −120 mV for 50 ms (to completely recover from inactivation, however channels that had drugs bound to them will not recover from inactivation) and this pattern was repeated 20 times with a sweep interval of 100 ms (10 Hz). Each concentration of compound was applied for 2 minutes. Experiments were performed at room temperature (approximately 22° C.).

### Ion Channel Target Profiling—Results and Data Analysis

Only current amplitudes in excess of 200 pA at the control stage were analyzed. The amplitude of the hNav1.1 to hNav1.8 current was calculated by measuring the difference between the peak inward current on stepping to −10 mV (i.e. peak of the current) and remaining current at the end of the step. The hNav1.1 to hNav1.8 currents were assessed in vehicle control conditions and then at the end of each two (2) minute compound application. Individual cell trap results were normalized to the vehicle control amplitude. These values were then plotted and estimated IC₅₀ curve fits calculated.

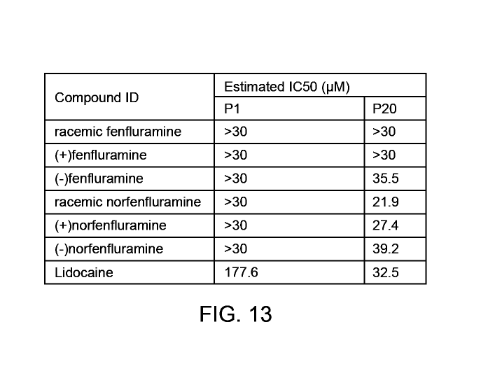

IC₅₀ values calculated for hNav1.5 are shown in FIG. 13. Results obtained for the remaining receptors did not show significant activity, and are therefore not shown.

### Example 6

### Sigma-1 Receptor Tissue Function Bioassay

Activity of the test compounds at Sigma-1 receptors was measured using a guinea pig vas deferens tissue bioassay. See Vaupel D. B. and Su T. P. (1987), Guinea-pig vas deferens preparation can contain both sigma and phencyclidine receptors, Eur. J. Pharmacol., 139: 125.

### Sigma-1 Receptor Tissue Bioassay—Materials and Methods

### Sigma-1 Receptor Tissue Bioassay—Tissue Preparation

Segments of guinea pig vas deferens were suspended in 20-ml organ baths containing an oxygenated (95% O2 and 5% CO2) and pre-warmed (37° C.) physiological salt solution of the following composition (in mM): NaCl 118.0, KCl 4.7, MgSO4 1.2, CaCl2 2.5, KH2PO4 1.2, NaHCO3 25 and glucose 11.0 (pH 7.4). Yohimbine (1 μM), (−)sulpiride (1 μM), atropine (1 μM), naloxone (1 μM), propanolol (1 μM), cimetidine (1 μM) and methysergide (1 μM) were also present throughout the experiments to block the alpha-2-adrenergic, beta-adrenergic, dopamine D2, histamine, muscarinic, 5-HT2, 5-HT3 and 5-HT4 serotonin and opioid receptors, respectively.

The tissues were connected to force transducers for isometric tension recordings. They were stretched to a resting tension of 0.5 g then allowed to equilibrate for 60 min during which time they were washed repeatedly and the tension readjusted. Thereafter, they were stimulated electrically with 1-sec trains of square wave pulses (maximal intensity, 1 msec duration, 5 Hz) delivered at 10-sec intervals by a constant current stimulator.

The experiments were carried out using semi-automated isolated organ systems possessing eight organ baths, with multichannel data acquisition.

### Sigma-1 Receptor Tissue Bioassay—Agonist Activity

The tissues were exposed to a submaximal concentration of the reference agonist (+)SKF-10047 (100 μM) to verify responsiveness and to obtain a control response.

Following washings and recovery of the initial twitch contractions, the tissues were exposed to increasing concentrations of the test compound or the same agonist. The different concentrations were added cumulatively and each left in contact with the tissues until a stable response was obtained or for a maximum of 15 min.

Where an agonist-like response (enhancement of twitch contractions) was obtained, the reference antagonist rimcazole (10 μM) was tested against the highest concentration of the compound to confirm the involvement of the sigma receptors in this response.

### Sigma Receptor—Antagonist Activity Assay

The tissues were exposed to a submaximal concentration of the reference agonist (+)SKF-10047 (100 μM) to obtain a control response.

After stabilization of the (+)SKF-10047-induced response, increasing concentrations of the test compound or the reference antagonist rimcazole were added cumulatively. Each concentration was left in contact with the tissues until a stable response was obtained or for a maximum of 15 min.

If it occurred, an inhibition of the (+)SKF-10047-induced increase in twitch contraction amplitude by the test compound indicated an antagonist activity at the sigma receptors.

### Sigma-1 Receptor Tissue Bioassay—Data Analysis and Results

Results, expressed as a percent of the control agonist response, are shown in FIG. 9B. When at least 6 compound concentrations were tested, the EC₅₀ value (concentration producing a half-maximum response) or IC₅₀ value (concentration causing a half-maximum inhibition of the response to the reference agonist) was determined by linear regression analysis of the concentration-response curves.

In the field-stimulated guinea pig vas deferens, the receptor agonist (+)SKF-10,047 induced a concentration-dependent increase in the twitch contraction amplitude, which was inhibited by the antagonist rimcazole in a concentration-dependent manner.

Racemic fenfluramine and its enantiomers did not significantly affect twitch contraction amplitude but slightly increased the (+)SKF10,047-induced increase in the twitch contraction amplitude. Racemic norfenfluramine and (+) norfenfluramine induced a concentration-dependent decrease in twitch contraction amplitude whereas (−) norfenfluramine triggered a more complex behavior. Racemic norfenfluramine and its enantiomers induced a concentration-dependent inhibition of the (+)SKF-10,047-induced increase in the twitch contraction amplitude.

### Sigma-1 Receptor Tissue Bioassay—Conclusions

These results support the conclusion that racemic fenfluramine and its enantiomers behave as positive allosteric modulators of the sigma receptor, whereas racemic norfenfluramine and its enantiomers behave as inverse agonists. Activity of the latter compounds in the agonist effect assay can indicate a more complex behavior involving other receptors.

### Example 1 Through 6

### Binding and Functional Assays Summary Results and Conclusions

### Summary Results—Binding Assays

Results of the initial receptor binding assays described in Example 1 are shown in FIG. 1A and FIG. 1B. Those results show that racemic fenfluramine and racemic norfenfluramine show moderate to strong binding to the 5-HT1A receptor, the β adrenergic receptor, the β2 adrenergic receptor, the muscarinic M1 receptor, the Nav 1.5 ion channel subunit, and the sigma-1 receptor.

Results of the binding studies comparing the binding activities of racemic fenfluramine and norfenfluramine, as described in Example 2, are shown in FIG. 2. Those results also demonstrate that racemic fenfluramine and racemic norfenfluramine show moderate to strong binding to the β adrenergic, β2 adrenergic, muscarinic M1, Na channel, 5-HT1A, and sigma receptors.

A comparison of the binding activities of fenfluramine and norfenfluramine enantiomers, as described in Example 3 are shown in FIG. 4. These results demonstrate that, for the receptors tested, there is little or no difference in binding activity as between the enantiomers of either fenfluramine or norfenfluramine.

### Summary Results—Functional Assays

Summary Results of the functional activity assays for the 5-HT1A receptor, the beta-2 adrenergic receptor, the described in Example 4 and Example 5 are shown in FIG. 8. These results demonstrated the following:

None of the test compounds had either agonist or antagonist activity at the 5-HT1A receptor.

Both racemic fenfluramine and norfenfluramine had some antagonist activity at the beta-2 adrenergic receptor, while racemic norfenfluramine acted as a weak antagonist at both the sigma receptor and the Nav1.5 ion channel receptor. Enantiomers of fenfluramine and norfenfluramine did not, for the most part, differ in binding activity at any of those receptors.

There were some differences between the activities of the enantiomers at the muscarinic M1 receptor, where the (+)-fenfluramine and the (−) norfenfluramine enantiomers showed some antagonist activity, while the corresponding enantiomers did not. That difference was not large, representing only one order of magnitude in concentration.

Finally, the results of the sigma tissue assay described in Example 5 are consistent with the conclusion that racemic fenfluramine and its enantiomers behave as positive allosteric modulators of the sigma receptor, whereas racemic norfenfluramine and its enantiomers behave as inverse agonists. Activity of the latter compounds in the agonist effect assay can indicate a more complex behavior involving other receptors.

### Example 7

### Sigma-1 and 5-HT Components of Fenfluramine and Norfenfluramine's Pharmaceutical Effects

The mechanism underlying the pharmacological effects of fenfluramine and norfenfluramine and their stereoisomers (collectively, the “test compounds”) were investigated in a series of three experiments in Swiss OF-1 mice. One experiment examined interaction of the 5-HT1A and sigma-1 receptor. A second experiment tested positive allosteric modulator activity of the test compounds on sigma-1 receptor activity.

### Sigma-1 and 5-HT Activity—Materials and Methods

### Animals

Male Swiss OF-1 mice, aged 7-9 weeks and weighing 32±2 g were purchased from Janvier (St Berthevin, France). Mouse housing and experiments took place within the animal facility of the University of Montpellier (CECEMA, registration number D34-172-23). Animals were housed in groups with access to food and water ad libitum. They were kept in a temperature and humidity controlled facility on a 12 h/12 h light/dark cycle (lights on at 7:00 h).

Behavioral experiments were carried out between 9:00 h and 17:00 h, in a sound attenuated and air-regulated experimental room, to which mice were habituated for 30 min. All animal procedures were conducted in strict adherence to the European Union Directive of Sep. 22, 2010 (2010/63).

### Drugs and Injections

The reagents employed in the experiments described in this example, along with their IUPAC names (as appropriate), and the sources from which they were obtained, are tabulated below.

TABLE 4
Sources for Reagents

| Agent | IUPAC name | Activity |
|---|---|---|
| WAY-10065 | N-[2-[4-(2-Methoxyphenyl)-1- | 5-HT1A Selective |
| | piperazinyl]ethyl]-N-2- | antagonist; |
| | pyridinylcyclohexanecarboxamide | Full D4 agonist |
| | maleate salt | |
| S(−)-8- | 7-(Dipropylamino)-5,6,7,8- | 5-HT1A full |
| hydroxy-DPAT | tetrahydronaphthalen-1-ol | agonist |
| hydrobromide | hydrobromide | |
| (8-OH-DPAT) | | |
| RS-127445 | 2-Amino-4-(4-fluoronaphth- | 5-HT2B selective |
| | 1-yl)-6-isopropylpyrimidine | antagonist |
| | hydrochloride | |
| SB 242084 | 6-Chloro-2,3-dihydro-5-methyl- | 5-HT2C selective |
| | N-[6-[(2-methyl-3- | antagonist |
| | pyridinyl)oxy]-3-pyridinyl]- | |
| | 1H-indole-1-carboxyamide | |
| | dihydrochloride hydrate | |
| GR 127935 | N-[4-Methoxy-3-(4-methyl-1- | selective |
| | piperazinyl)phenyl]-2′- | antagonist of |
| | methyl-4′-(5-methyl-1,2,4- | 5-HT1B and |
| | oxadiazol-3-yl)-1,1′-biphenyl-4- | 5-HT1D |
| | carboxamide hydrochloride hydrate | |
| igmesine | (R)-(+)-N-Cyclopropylmethyl-α- | Sigma receptor |
| | ethyl-N-methyl-α-[(2E)-3-phenyl- | agonist |
| | 2-propenyl)benzenemethanamine | |
| | hydrochloride | |
| PRE-084 | 2-(4-Morpholinethyl)-1- | sigma-1 selective |
| | phenylcyclohexanecarboxylate | agonist |
| | hydrochloride | |
| NE-100 | 4-Methoxy-3-(2-phenylethoxy)-N,N- | Selective sigma-1 |
| | dipropylbenzeneethanamine | antagonist |
| | hydrochloride | |
| (+)-MK-801 | (5S,10R)-(+)-5-Methyl-10,11- | Impairs learning |
| (dizocilpine) | dihydro-5H-dibenzo[a,d]cyclohepten- | |
| | 5,10-imine hydrogen maleate | |

All drugs were solubilized in physiological saline (vehicle solution) and administered intraperitoneally (IP), in a volume of 100 μl per 20 g body weight.

### Forced Swim Test

The forced swim test (“FST”) assesses behavioral despair in mice. Previously, the FST has been used as a model system for testing the efficacy of putative antidepressants. Prior reports have provided evidence that behavioral despair is mediated by the same receptor types implicated in fenfluramine's mechanism of action (see Examples 1 through Example 6 herein). It was used here as a behavioral assay to investigate whether the same receptors implicated in fenfluramine binding and functional activity, and its in vivo anti-epileptiform effects in mutant zebrafish (see Examples 1 though Example 6 and Example 8), also mediate its biological effects in mammals. See Urani et al., 2001; Villard et al., 2011).

On day 1, each mouse was placed individually in a glass cylinder (diameter 12 cm, height 24 cm) filled with water at a height of 12 cm. Water temperature was maintained at 23±1° C. Animals were forced to swim for 15 min and then returned to their home cage. On day 2, animals were placed again into the water and forced to swim for 6 min. The mouse was considered as immobile when it stopped struggling and moved only to remain floating in the water, keeping its head above water. The session was video-tracked (Viewpoint, Lisieux, France) and the quantity of movement quantified min per min by the software. The duration of immobility was analyzed during the last 5 min of the after returning the mouse to its home cage. None of the animals included in the study exhibited a particular hypomobility response due to hypothermia; however, direct measure of hypothermia was not performed. Drugs were administered on the second day 30′ prior to the swim session.

### Spontaneous Alternation in the Y Maze

Animals were tested for spontaneous alternation performance in the Y-maze, an index of spatial working memory (Maurice et al., 1994a,b, 1998; Meunier et al., 2006; Maurice, 016).

The Y-maze is made of grey PVC. Each arm is 40 cm long, 13 cm high, 3 cm wide at the bottom, 10 cm wide at the top, and converged at an equal angle. Each mouse was placed at the end of one arm and allowed to move freely through the maze during an 8-min session. The series of arm entries, including possible returns into the same arm, were checked visually. An alternation was defined as entries into all three arms on consecutive occasions. The number of maximum alternations was therefore the total number of arm entries minus two and the percentage of alternation was calculated as:

actual alternations/maximum alternations)×100.

Parameters included the percentage of alternation (memory index) and total number of arm entries (exploration index).

### Step-Through Passive Avoidance

The test assesses non-spatial/contextual long-term memory and was performed as previously described (Meunier et al., 2006; Maurice, 2010). The apparatus consisted of a 2-compartment box, with one illuminated with white polyvinylchloride walls and a transparent cover (15×20×15 cm high), one with black polyvinylchloride walls and cover (15×20×15 cm high), and a grid floor. A guillotine door separated each compartment. A 60 W lamp was positioned 40 cm above the apparatus lit the white compartment during the experimental period.

Scrambled foot shocks (0.3 mA for 3 s) were delivered to the grid floor using a shock generator scrambler (Lafayette Instruments, Lafayette, Mass., USA). The guillotine door was initially closed during the training session. Each mouse was placed into the white compartment. After 5 s, the door was raised. When the mouse entered the darkened compartment and placed all its paws on the grid floor, the door was gently closed and the 3 scrambled foot shock was delivered for 3 s. The step-through latency, i.e., the latency spent to enter the dark compartment, and the level of sensitivity to the shock were recorded. The latter was evaluated as: 0=no sign; 1=flinching reactions; 2=flinching and vocalization reactions. The retention test was carried out 24 h after training. Each mouse was placed again into the white compartment. After 5 s, the door was raised. The step-through latency was recorded up to 300 s. Animals entered the darkened compartment or were gently pushed into it and the escape latency, i.e., the time spent to return into the white compartment, was also measured up to 300 s. Results were expressed as median and interquartile (25%-75%) range.

### Statistical Analyses

Data were analyzed using a one-way analysis of variance (ANOVA, F value), followed by a Dunnett's test or a Kruskal-Wallis non-parametric ANOVA (H value). Passive avoidance latency data were additionally subjected to a third analysis step using Dunn's multiple comparison tests (expressed as median and interquartile range. The level of statistical significance was p<0.05.

### Combination Index Calculations

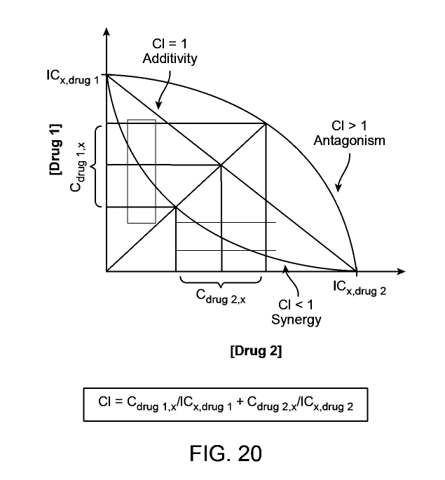

The isobologram analyses, evaluating the nature of interaction of two drugs at a given effect level were performed according to Fraser's concept (1872), Zhao et al. (2010) and Maurice 2016). A schematic of the isobologram plot used in combination index calculations is shown in FIG. 20.

Theoretically, the concentrations required to produce the given effect (e.g., IC₅₀) are determined for drug A (IC_{x,A}) and drug B (IC_{x,B}) and indicated on the x and y axes of a two-coordinate plot, forming the two points (IC_{x,A}, 0) and (0, IC_{x,B}). The line connecting these two points is the line of additivity. Then, the concentrations of A and B contained in the combination that provides the same effect, denoted as (C_{A,x}, C_{B,x}), are placed in the same plot. Synergy, additivity, or antagonism is indicated when (C_{A,x}, C_{B,x}) is located below, on, or above the line, respectively. Operationally, a combination index (CI) is calculated as:

CI=CA,x/ICx,A+CB,x/ICx,B

where C_{A/B,x} are the concentrations of drug A/B used in a combination that generates x % of the maximal combination effect; CI is the combination index; and IC_{x,A/B} is the concentration of drug A/B needed to produce x % of the maximal effect. A CI of less than, equal to, and more than 1 indicates synergy, additivity, and antagonism, respectively.

### 7(A)

### Additive or Synergistic Effects of the 5-HT1A and Sigma-1 Receptors

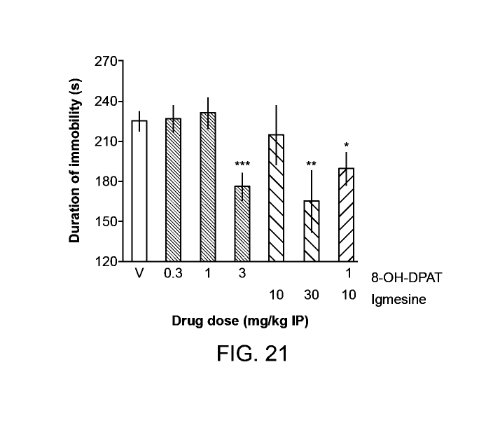

8-OH-DPAT, a 5-HT1A receptor agonist, and the sigma-1 receptor agonist igmesine were tested alone and in combination in Swiss mice subjected to the FST. Animals were treated IP with Igmesine at 10 mg/kg or 30 mg/kg, 8-OH-DPAT at 0.3 mg/kg, 1 mg/kg, or 3 mg/kg, or with a combination of 10 mg/kg Igmesine and 1 mg/kg 8-OH-DPAT. Results are presented in FIG. 21 as the mean±SEM of the number of animals (n).

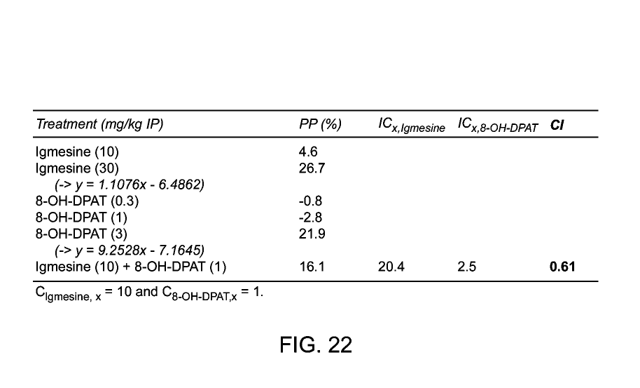

In order to perform the isobologram derived calculation (i.e., calculation of combination index CI), durations of immobility were expressed as percentage of protection (PP) for each treatment group, considering that PP (zero immobility)=100% and PP (V-treated group)=0%. The PP were calculated for the group and the combination and are shown in FIG. 22. For each drug, the linear regression was estimated, the C_{x,drug} determined, and the CI calculated as above.

The acute IP injection of 8-OH-DPAT reduced immobility in Swiss mice submitted to the FST at 3 mg/kg but not at the lower doses tested, 0.1, 0.3, 1 mg/kg IP (FIG. 21). The dose was found slightly higher than published previously by other authors: 0.25-0.5 mg/kg in male CD-COBS rats, in Cervo & Samanin (1987); 0.5 mg/kg in male Sprague-Dawley rats, in Singh & Lucki (1993); and 0.25-1 mg/kg in female BKTO mice, in O'Neill & Conway (2001). Igmesine decreased immobility duration at 30 mg/kg IP. Combination of the maximal non-active dose of 8-OH-DPAT and igmesine led to a significant reduction of the immobility duration.

Calculation of the combination index for this mix (FIG. 22) led to a CI=0.61, indicative of a synergy between the two drugs, which supports the conclusion that an interaction between thee 5-HT1A receptor and the sigma 1 receptor is implicated in fenfluramine's mechanism of action.

### 7(B)

### Combined Effects of Fenfluramine and Sigma 1 Agonists

Putative positive allosteric modulator (PAM) activity of fenfluramine on sigma-1 receptors was investigated by testing fenfluramine's ability to prevent the effects of dizocilpine (a potent anti-convulsant which negatively affects memory) in two complementary behavioral tests assessing short- and long-term memories, as described in the Materials and Methods section above.

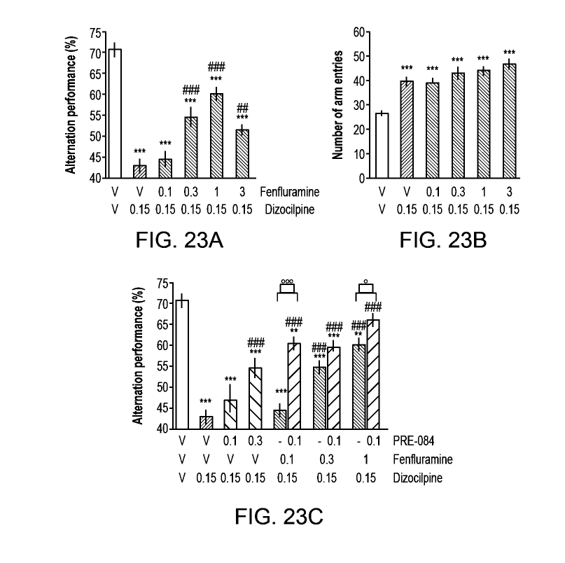

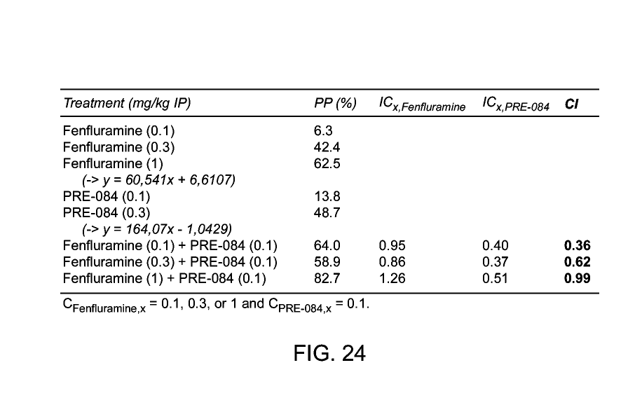

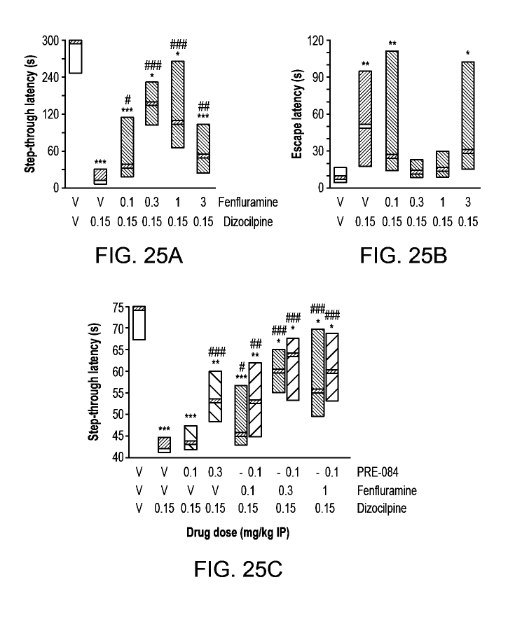

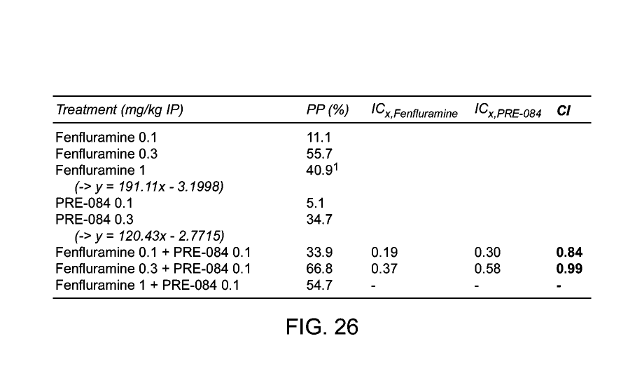

PRE-084 (a selective sigma 1 agonist) and fenfluramine were tested alone and in combination in Swiss mice. The drugs were administered IP with dizocilpine (0.15 mg/kg, and tested in the spontaneous alternation test on day 1 and in the passive avoidance test on days 2-3. Results are presented in FIG. 23A, FIG. 23B, FIG. 23C, FIG. 24, FIG. 25A, FIG. 25B, FIG. 25C, and FIG. 26.

Statistical analysis: Dose-response of fenfluramine: F(5,78)=16.79, p<0.0001 for Loc, F(5,78)=35.80, p<0.0001 for Alt %. Combination with PRE-084: F(9,127)=22.11, p<0.0001 for Alt %.

### Calculation of the Combination Index (CI)—Y-Maze Test

As a first step in performing the isobologram derived calculation, mean alternation percentages were expressed as percentage of protection (PP) for each treatment group, considering that PP (V-treated group)=100% and PP (Dizocilpine-treated group)=0%. The PP were calculated for each group and the combination and are shown in FIG. 26. The linear regression was estimated, the Cx,drug determined, and the CI calculated as previously, for each drug. Results are presented as median and interquartile [25%-75%] range and mean±SEM of the number of animals (n).

### Statistical Analyses:

Dose-response of fenfluramine (upper part of table shown in FIG. 24 and FIG. 26): Kruskal-Wallis ANOVA, using H=7.69, p>0.05 for STL-Tg, H=2.78, p>0.05 for SS-Tg, H=36.5, p<0.0001 for STL=13.1, and p<0.05 for EL-R. For the combination with PRE-084 (lower part of the table in FIG. 24 and FIG. 26): H=49.5, p<0.0001 for STL-R.

### Calculation of the Combination Index (CI)—Passive Avoidance Test

In order to perform the isobologram derived calculation, median step-through latencies were expressed as percentage of protection (PP) for each treatment group, considering that PP (V-treated group)=100% and PP (Dizocilpine-treated group)=0%. The PP were calculated for each group and the combination and are shown in FIG. 26. The linear regression was estimated, the Cx,drug determined, and the CI calculated as previously, for each drug. Note that the CI calculation could not include the Fenfluramine 1 mg/kg dose since the 0.1-1 data appeared far from linearity. The linear regression was therefore limited to the 0.1 (maximal non-active dose) and 0.3 (minimal active dose) doses, i.e., using C_{fenfluramine,x}=0.1, 0.3, or 1, and C_{PRE-084,x}=0.1

### Comments:

Dizocilpine administration in mice produced drastic alterations of spontaneous alternation (FIG. 23A) and passive avoidance learning (FIG. 25A). Fenfluramine racemate significantly attenuated both deficits and the most active doses appeared to be 0.3 and 1 mg/kg IP (FIG. 23A, FIG. 25A, and FIG. 25B). The drug did not affect dizocilpine-induced locomotor increase at these doses (FIG. 25B). The profile is highly coherent as could be expected from a sigma-1 acting drug (Maurice et al., 1994a, b). Co-administration of NE-100 with fenfluramine 0.3 or 1 mg/kg could help confirm the sigma-1 receptor involvement in the drug effect.

Combination studies were performed with PRE-084. As shown first, the reference sigma-1 agonist attenuated dizocilpine-induced deficits in spontaneous alternation and passive avoidance response at 0.3 but not 0.1 mg/kg IP (FIG. 4c, 5c), as previously described (Maurice et al., 1994b). Then, combination between PRE-084 (0.1) and increasing doses of fenfluramine (0.1, 0.3, 1) were tested. Combination indexes were calculated using the mean percentage of alternation or the median step-through latency.

For spontaneous alternation (FIG. 24), CI<1 for the (Fenfluramine 0.1+PRE-084 0.1) and (Fenfluramine 0.3+PRE-084 0.1) mix indicated a synergy between the two drugs. At the highest doses, the mix led to a CI=1 indicating an additivity between the two drugs.

For passive avoidance (FIG. 26), CI<1 for the (Fenfluramine 0.1+PRE-084 0.1) indicated a synergy between the two drugs. At the dose (Fenfluramine 0.3+PRE-084 0.1), the mix led to a CI=1 indicating an additivity between the two drugs.

### Example 7

### Conclusion

The data indicated a strong interaction between fenfluramine and PRE-084. Particularly, fenfluramine, at its maximal inactive dose (0.1) is able to synergistically boost PRE-084 effect. This data supports the conclusion that fenfluramine behaves as a sigma-1 receptor PAM.

### Example 7

### REFERENCES

Cervo L, Samanin R. Potential antidepressant properties of 8-OH-Dpat (8-hydroxy-2-(di-N-propylamino) tetralin, a selective serotonin1A receptor agonist. Eur J Pharmacol. 1987; 144: 223-9.

Fraser T R. The antagonism between the actions of active substances. Br Med J. 1872; 485-87.

Maurice T, Hiramatsu M, Itoh J, Kameyama T, Hasegawa T, Nabeshima T. Behavioral evidence for a modulating role of sigma ligands in memory processes. I. Attenuation of dizocilpine (MK-801)-induced amnesia. Brain Res. 1994a; 647: 44-56.

Maurice T, Su T P, Parish D W, Nabeshima T, Privat A. PRE-084, a sigma selective PCP derivative, attenuates MK-801-induced impairment of learning in mice. Pharmacol Biochem Behav. 1994b; 49: 859-69.

Maurice T, Su T P, Privat A. Sigmal receptor agonists and neurosteroids attenuate β25-35-amyloid peptide-induced amnesia in mice through a common mechanism. Neuroscience. 1998; 83: 413-28.

Maurice T. Protection by sigma-1 receptor agonists is synergic with donepezil, but not with memantine, in a mouse model of amyloid-induced memory impairments. Behav Brain Res. 2016; 296: 270-278.

Meunier J, Ieni J, Maurice T. The anti-amnesic and neuroprotective effects of donepezil against amyloid β25-35 peptide-induced toxicity in mice involve an interaction with the sigma-1 receptor. Br J Pharmacol. 2006; 149: 998-1012.

O'Neill M F, Conway M W. Role of 5-HT1A and 5-HT1B receptors in the mediation of behavior in the forced swim test in mice. Neuropsychopharmacology. 2001; 24: 391-8.

Singh A, Lucki I. Antidepressant-like activity of compounds with varying efficacy at 5-HT1A receptors. Neuropharmacology. 1993; 32: 331-40

Urani A, Roman F J, Phan V L, Su T P, Maurice T. The antidepressant-like effect induced by sigmal-receptor agonists and neuroactive steroids in mice submitted to the forced swimming test. J Pharmacol Exp Ther. 2001; 298: 1269-79.

Villard V, Meunier J, Chevallier N, Maurice T. Pharmacological interaction with the sigmal (sigma-1) receptor in the acute behavioral effects of antidepressants. J Pharmacol Sci. 2011; 115: 279-92.

Zhao L, Au J L, Wientjes M G. Comparison of methods for evaluating drug-drug interaction. Front Biosci. 2010; 2: 241-9.als

### Example 8

### Anti-Seizure Effects of Fenfluramine in 4 Animal Models of Refractory Epilepsies

The efficacy of fenfluramine (FA) in treating other seizures and other epilepsy syndromes was assessed in four animal model systems: (1) homozygous scn1Lab−/− mutant zebrafish (ZF), used as a genetic model of Dravet syndrome (DS); (2) a ZF model of pentylenetetrazole (PTZ)-induced seizures, a chemical model of kindling, (3) a mouse model of refractory seizure model, and (4) a mouse model of seizure-induced respiratory arrest.

### 8(A) and (B)

### Zebrafish Studies

FA treatment significantly decreased epileptiform behavior in homozygous scn1Lab−/− mutants (One-way ANOVA; p<0.05 vs. vehicle-treated (control), VHC). This anti-epileptiform activity was consistently confirmed by LFP recordings (Mann-Whitney test; p<0.05 vs. VHC). In addition, a concentration-dependent effect was observed, with more pronounced anticonvulsant activities observed at higher concentrations of FA.

ZF larvae were treated on 6 dpf with vehicle (VHC, 0.1% dimethyl sulfoxide, DMSO, or FA (25, 50 or 100 μM) for 24 h. Thereafter, the locomotor activity (behavior) was monitored by an automated tracking device. Subsequently, forebrain local field potentials and forebrain activity (LFPs, brain activity) were measured to confirm anticonvulsant effects of FA treatment, if indicated by the behavioral assays.

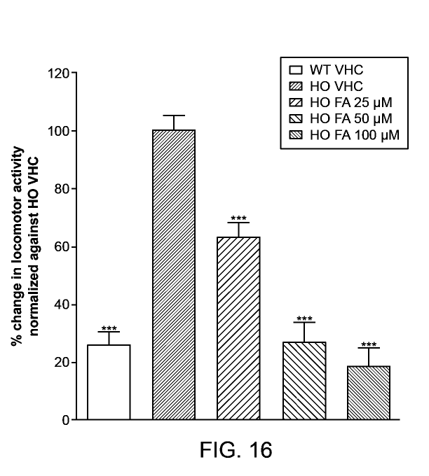

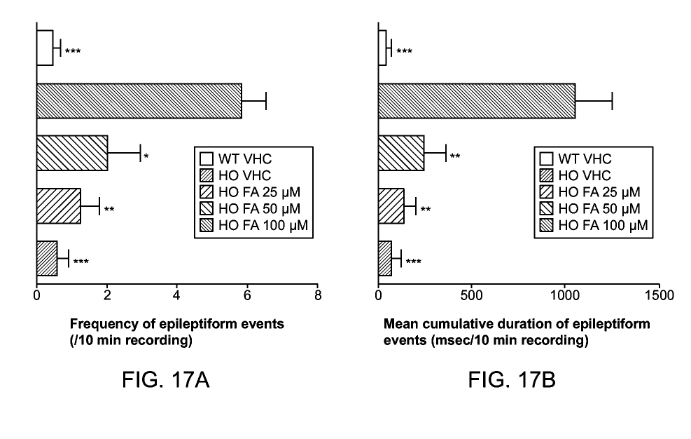

Results are shown in FIG. 16, FIG. 17A, and FIG. 17B. FA treatment significantly decreased epileptiform behavior in homozygous scn1Lab−/− mutants (One-way ANOVA; p<0.05 vs. vehicle-treated (control), VHC). This anti-epileptiform activity was consistently confirmed by LFP recordings (Mann-Whitney test; p<0.05 vs. VHC). In addition, a concentration-dependent effect was observed, with more pronounced anticonvulsant activities observed at higher concentrations of FA.

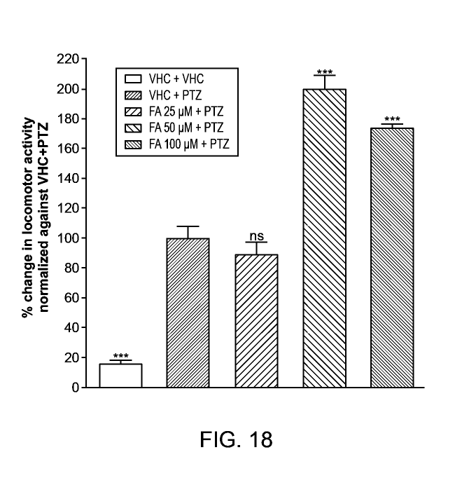

In contrast, fenfluramine had no observable antiseizure effects in wt zebrafish when given following seizure induced with PTZ See FIG. 18 (One-way ANOVA; p>0.05 vs. VHC+PTZ

### 8(C)

### 6 Hz Mouse Studies

The 6 Hz mouse model is a model system used to assess the efficacy of putative anti-seizure medications for refractory epilepsies generally, without regard to type.

The efficacy of FA in the mouse 6-Hz model was assessed by behavioral characterization after intraperitoneal injection of CRL NMRI mice (30-35 g) with VHC (50/50 DMSO/PEG200) or FA (5.0 or 20.0 mg/kg). Animals were placed in one of three treatment groups: vehicle (n=10), FA 5 mg/kg (n=6) and FA 20 mg/kg (n=6). Seizures were transcorneal-induced 1 h after injection (6-Hz, 0.2 ms pulse width, 44 mA).

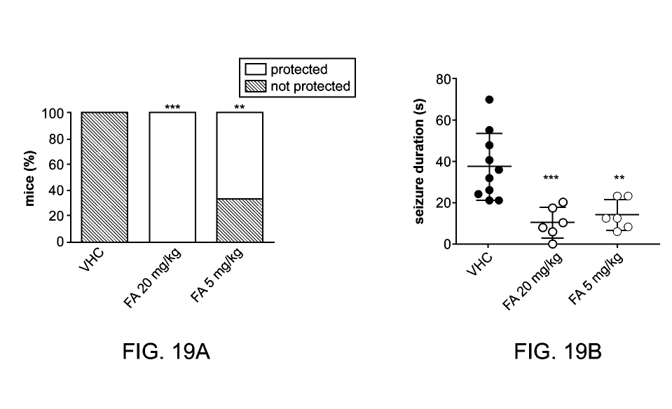

Results are shown in FIGS. 19A and 19B. Fenfluramine significantly reduced seizures in the mouse 6-Hz model (Mann-Whitney test; p<0.05 vs. VHC). A dose-dependent decrease in number of mice having seizures and in duration of seizures was observed. Additionally, the mice injected with vehicle displayed a period of post-seizure aggression, whereas the mice treated with fenfluramine did not.

Thus, fenfluramine is effective in reducing seizures in an animal model of refractory epilepsies other than Dravet syndrome.

### 8(D)

### Effects of Fenfluramine on Audiogenic Seizures and Seizure-Induced Respiratory Arrest (S-IRA) in DBA/1 Mice

Prevention of premature mortality due to sudden unexpected death in epilepsy (SUDEP) is a major goal in epilepsy. Most of the witnessed clinical cases reported generalized seizures leading to respiratory and cardiac failure leading to SUDEP. The DBA/1 mouse model of SUDEP exhibits generalized tonic-clonic seizures resulting in S-IRA, which leads to cardiac arrest and death. The inventors previously found that several selective serotonin (5-HT) reuptake inhibitors prevent S-IRA in DBA mice. However, not all the drugs that enhance the activation of 5-HT receptors effectively block S-IRA in DBA mice. Therefore, the present study investigated if ±fenfluramine (FFA), which augments 5-HT release, alters susceptibility to audiogenic seizures and S-IRA in DBA/1 mice.

### 8(D)

### Materials and Methods

DBA/1 mice (21-30 days old) were primed by being subjected to audiogenic seizures and S-IRA with 3-4 seizures (once daily), using an electrical bell. Mice that consistently showed S-IRA susceptibility on 3 consecutive tests and were resuscitated with a rodent respirator were studied. At least 24 h after priming, the mice received either FFA (5-40 mg/kg) or saline (vehicle) intraperitoneally and were tested for susceptibility to seizures and S-IRA. Seizure behaviors were recorded on videotape, quantified, and compared statistically (Chi-Square Test; significance set at p<0.05).

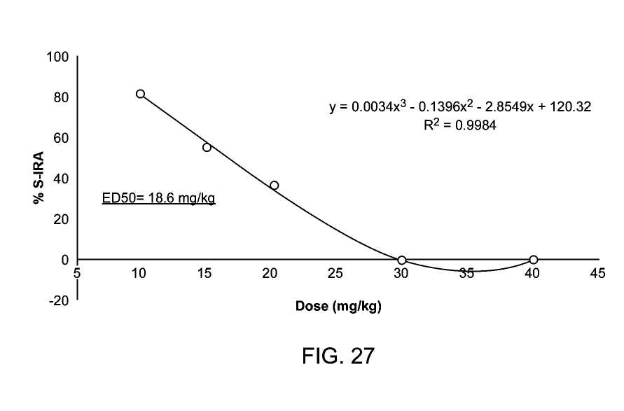

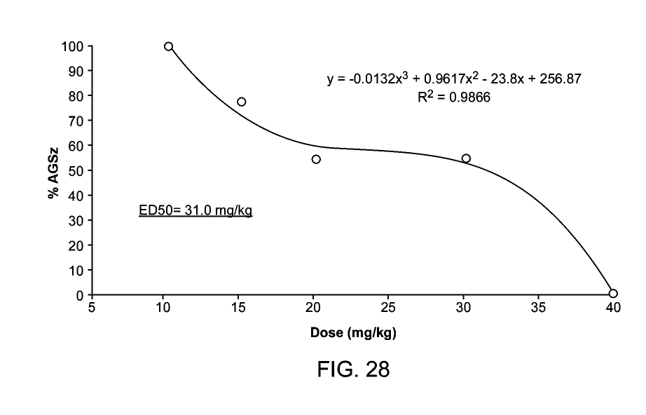

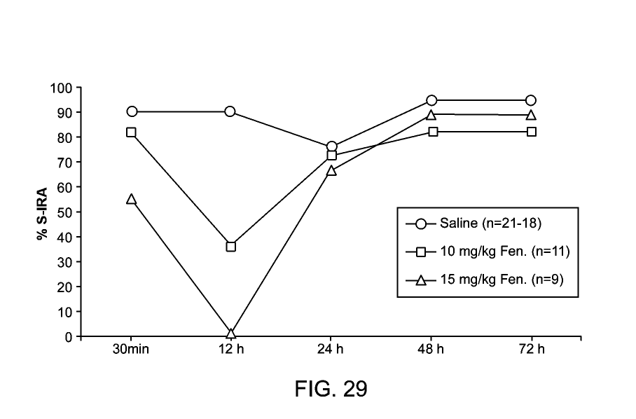

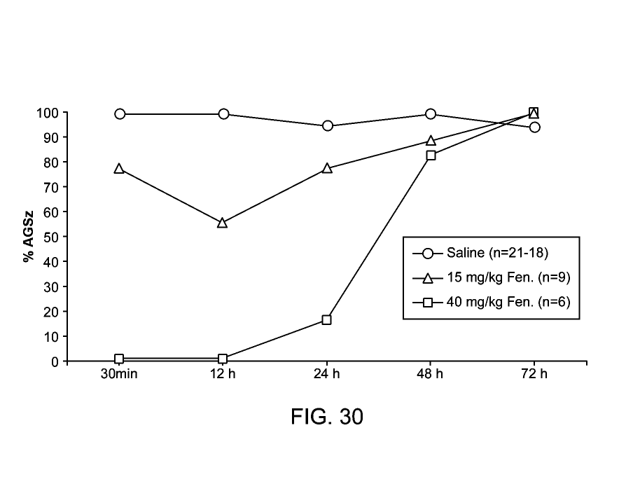

Administration of fenfluramine Intraperitoneally (i.p.) in DBA/1 mice resulted in a dose-dependent blockade of seizure-induced respiratory arrest (S-IRA). The ED50 for this effect was 18.6 mg/kg at 30 min post drug (See FIG. 27) as compared to fluoxetine (22.2 mg/kg). Higher doses of fenfluramine also resulted in a dose-dependent blockade of susceptibility to audiogenic seizures (AGSz). The ED50 for this effect was 31.0 mg/kg at 30 min. See FIG. 28). The time course of these effects of this acute fenfluramine administration was prolonged, lasting at least 24 h and sometimes as long as 72 h in some mice, depending on dose (FIG. 29 and FIG. 30).

Results: We characterized the dose-response relationship for FFA against seizures and S-IRA in DBA/1 mice by testing susceptibility at 30 min, 12 and 24 h, and then at 24 h intervals. Mice that received 10 (n=11) and 15 mg/kg (n=9) of FFA showed significantly (p<0.01) reduced S-IRA susceptibility and seizure severity at 12 h. The 40 mg/kg dose of FFA (n=6) completely blocked seizures (p<0.001) at 30 min, and seizure and S-IRA susceptibility returned at 48 and 72 h, respectively. The ED50 value of FFA against seizure susceptibility at 30 min was 21.4 mg/kg. A more detailed study of the time course of effect was done using 5 (n=9), 10 (n=10), 15 (n=10) and 20 mg/kg (n=9) doses of FFA at 8 h intervals over a 24 h period. We found that 15 mg/kg showed a significantly reduced seizure severity (p<0.05) and a selective S-IRA blocking effect (p<0.001) at 16 h. A reduction in S-IRA incidence and seizure severity by the 10-20 mg/kg doses of FFA occurred at 8 h. The 5 mg/kg dose was ineffective. The susceptibility to seizure and S-IRA returned by 48 h after FFA treatment.

### 8(D)

### Conclusions

Collectively, the data from these animal models provide proof of principle for the use of fenfluramine as an anti-seizure mediation for treating refractory seizures in addition to Dravet syndrome.

FFA was effective in blocking S-IRA and seizures in DBA/1 mice in a dose- and time-dependent manner. Blockade of S-IRA by FFA was long-lasting unlike that of all other 5-HT-enhancing drugs previously tested. Our studies are the first to show the efficacy of FFA in a mammalian model of SUDEP. This data is proof of principle for FFA's efficacy in the prophylaxis of SUDEP, which is in addition to its effects in improving seizure control, and is relevant toward explaining the underlying mechanism of the recent success of FFA in treatment of Dravet Syndrome patients who have a high risk of SUDEP (Ceulemans et al., Epilepsia, 2016). This research is supported by a grant from Zogenix Inc.

### Example 9

### High-Throughput Screening of Candidate Therapeutic Agents

As a first step in identifying novel therapeutic agents, compounds provided by the present disclosure are assessed for their anticonvulsant activity in vitro using the high-throughput mutant zebrafish screening assay of Zhang et al., as described in ACS Nano, 2011, 5 (3), pp 1805-1817; DOI: 10.1021/nn102734s, e-published on Feb. 16, 2011.

### Test Compounds

Compounds for drug screening are provided as 10 mM DMSO solutions. Test compounds for locomotion or electrophysiology studies are dissolved in embryo media and are tested at an initial concentration of 100 M, with a final DMSO concentration of 2%. In all drug screen studies, compounds are coded and experiments are performed by investigators who are blind to the nature of the compound.

Drug concentrations between 0.5 and 1 mM are used for electrophysiology assays to account for more limited diffusion in agar-embedded larvae.

### Animals

Zebrafish are maintained in a light- and temperature-controlled aquaculture facility under a standard 14:10 h light/dark photoperiod. Adult Heterozygous scn1Lab± mutant zebrafish (originally a gift from Dr. H. Baie, Freiburg, Germany and available commercially) are housed in 1.5 L tanks at a density of 5-12 fish per tank and fed twice per day (dry flake and/or flake supplemented with live brine shrimp). Water quality is continuously monitored to maintain the following conditions: temperature, 28-30° C.; pH 7.4-8.0; conductivity, 690-710 mS/cm. Zebrafish embryos are maintained in round Petri dishes (catalog #FB0875712, Fisher Scientific) in “embryo medium” consisting of 0.03% Instant Ocean (Aquarium Systems, Inc.) and 000002% methylene blue in reverse osmosis-distilled water.

Larval zebrafish clutches are bred from wild-type (WT; TL strain) or scn1Lab (didys552) heterozygous animals that had been back-crossed to TL wild-type for at least 10 generations. Homozygous mutants (n 6544), which have widely dispersed melanosomes and appear visibly darker as early as 3 d post-fertilization, or WT larvae (n=71) are used in all experiments at 5 or 6 dpf. Embryos and larvae are raised in plastic petri dishes (90 mm diameter, 20 mm depth) and density is limited to 60 per dish. Larvae between 3 and 7 dpf lack discernible sex chromosomes. The care and maintenance protocols comply with requirements [outlined in the Guide for the Care and Use of Animals (ebrary Inc., 2011) and are subject to approval by the Institutional Animal Care and Use Committee (protocol #AN108659-01D)].

### Seizure Monitoring

Zebrafish larvae are placed individually into 1 well of a clear flat-bottomed 96-well microplate (catalog #260836, Fisher Scientific) containing embryo media.

To study changes in locomotion, microplates are placed inside an enclosed motion-tracking device and acclimated to the dark condition for 10-15 min at room temperature. Locomotion plots are obtained for one fish per well at a recording epoch of 10 min using a DanioVision system running EthoVision XT software (DanioVision, Noldus Information Technology); threshold detection settings to identify objects darker than the background are optimized for each experiment. Seizure scoring is performed using the following three-stage scale (Baraban et al., 2005): Stage 0, no or very little swim activity; Stage I, increased, brief bouts of swim activity; Stage II, rapid “whirlpool-like” circling swim behavior; and Stage III, paroxysmal whole-body clonus-like convulsions, and a brief loss of posture. WT fish are normally scored at Stage 0 or I. Plots are analyzed for distance traveled (in millimeters) and mean velocity (in millimeters per second). As reported previously (Winter et al., 2008; Baraban et al., 2013), velocity changes are a more sensitive assay of seizure behavior.

For electrophysiology studies, zebrafish larvae are briefly paralyzed with bungarotoxin (1 mg/ml) and immobilized in 1.2% agarose; field recordings are obtained from forebrain structures. Epileptiform events are identified post hoc in Clampfit (Molecular Devices) and are defined as multi-spike or polyspike upward or downward membrane deflections greater than three times the baseline noise level and 500 ms in duration. During electrophysiology experiments zebrafish larvae are continuously monitored for the presence (or absence) of blood flow and heart beat by direct visualization on an Olympus BX51WI upright microscope equipped with a CCD camera and monitor.

Baseline recordings of seizure behavior are obtained from mutants bathed in embryo media, as described above; a second locomotion plot is then obtained following a solution change to a test compound and an equilibration period of 15-30 min. Criteria for a positive hit designation are as follows: (1) a decrease in mean velocity of 44% (e.g., a value based on the trial-to-trial variability measured in control tracking studies; FIG. 1c in Zhang et al.); and (2) a reduction to Stage 0 or Stage I seizure behavior in the locomotion plot for at least 50% of the test fish. Each test compound classified as a “positive hit” in the locomotion assay is confirmed, under direct visualization on a stereomicroscope, as the fish being alive based on movement in response to external stimulation and a visible heartbeat following a 60 min drug exposure.

Toxicity (or mortality) is defined as no visible heartbeat or movement in response to external stimulation in at least 50% of the test fish. Hyperexcitability is defined as a compound causing a 44% increase in swim velocity and/or Stage III seizure activity in at least 50% of the test fish. Hits identified in the primary locomotion screen are selected and rescreened, again using the method described above. Select compound stocks that are successful in two primary locomotion assays, and are not classified as toxic in two independent clutches of zebrafish, are then subjected to further testing.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

We claim:

1. A method for treating a patient in need of treatment comprising the step of administering an effective dose of a therapeutic agent, wherein the therapeutic agent comprises a compound active at one or more targets selected from the group consisting of:
(a) a 5-HT receptor protein selected from the group consisting of the 5-HT1A receptor, the 5-HT1D receptor, the 5-HT1E receptor, the 5-HT2A receptor, the 5-HT2C receptor, the 5-HT5A receptor, and the 5-HT7 receptor,
(b) an adrenergic receptor protein selected from the beta-1 adrenergic receptor, and the beta-2 adrenergic receptor,
(c) a muscarinic acetylcholine receptor protein selected from the group consisting of the M1 muscarinic acetylcholine receptor the M2 muscarinic acetylcholine receptor, the M3 muscarinic acetylcholine receptor, the M4 muscarinic acetylcholine receptor, and the M5 muscarinic acetylcholine receptor,
(d) a chaperone protein selected from the group consisting of the sigma-1 receptor and the sigma-2 receptor,
(e) a sodium channel subunit protein selected from the group consisting of the Nav 1.1 subunit, the Nav 1.2 subunit, the subunit, the Nav 1.3 subunit, the Nav 1.4 subunit, the Nav 1.5 subunit, the Nav 1.6 subunit, and the Nav 1.7 subunit, and
(f) a neurotransmitter transport protein selected from the group consisting of a serotonin transporter (SET), a dopamine transporter (DAT), and a norepinephrine transporter (NET).

2. The method of claim 1, wherein the therapeutic agent is a compound of Appendix 1.

3. The method of claim 1, wherein the therapeutic agent is active at one or more 5-HT receptor selected from the 5-HT1A receptor, the 5-HT1D receptor, the 5-HT2A receptor, and the 5-HT2C receptor.

4. The method as claimed in claim 1, wherein the therapeutic agent is a chaperone protein that is active at the sigma-1 receptor wherein the activity of the therapeutic agent is selected from the group consisting of positive allosteric modulation, allosteric agonism, positive ago-allosteric modulation, negative ago-allosteric modulation, and neutral ago-allosteric modulation.

5. The method as claimed in claim 4, wherein the therapeutic agent is a positive allosteric modulator.

6. The method of claim 1, wherein the therapeutic agent is active at to two or more targets or three or more targets.

7. The method of claim 6, wherein the therapeutic agent is active at the 5-HT1A receptor and further is active at the sigma-1 receptor.

8. The method of claim 6, wherein the therapeutic agent is active at one or more neurotransmitter transport proteins selected from the group consisting of SERT, DAT, and NET.

9. The method of claim 8, wherein the therapeutic agent is a compound according to the structure: 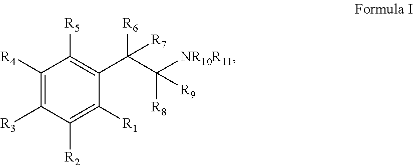
wherein
a. R1-R5 are each independently selected from H, OH, optionally substituted C1-4 alkyl, optionally substituted C1-3 alkoxy, optionally substituted C2-4 alkenyl, optionally substituted C2-4 alkynyl, halogen, amino, acylamido, CN, CF3, NO2, N3, CONH2, CO2R12, CH2OR12, NR12R13, NHCOR12, NHCO2R12, CONR12R13; C1-3 alkylthio, R12SO, R12SO2, CF3S, and CF3SO2;
b. R6 and R7 are each independently selected from H or optionally substituted C1-10alkyl, or R6 and R7 together constitute ═O or ═CH2;
c. R8 and R9 are each independently selected from H or optionally substituted C1-10alkyl;
d. R10, R11, R12, and R13 are each independently selected from H or optionally substituted C1-10 alkyl;
e. and wherein R1 and R8 may be joined to form a cyclic ring; or a pharmaceutically acceptable ester, amide, salt, solvate, prodrug, or isomer thereof,
with the proviso that when one of R8 and R9 is CH3, then at least one of R10 and R11 is optionally substituted C3-C10 cycloalkyl.

10. The method of claim 8, wherein the therapeutic agent is a compound according to the structure: 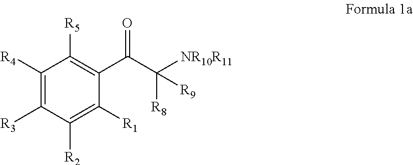
wherein
1. R1-R5 are each independently selected from H, OH, optionally substituted C1-4 alkyl, optionally substituted C1-3 alkoxy, optionally substituted C2-4 alkenyl, optionally substituted C2-4 alkynyl, halogen, amino, acylamido, CN, CF3, NO2, N3, CONH2, CO2R12, CH2OR12, NR12R13, NHCOR12, NHCO2R12, CONR12R13; C1-3 alkylthio, R12SO, R12SO2, CF3S, and CF3SO2;
2. R8 and R9 are each independently selected from H or optionally substituted C1-10 alkyl;
3. R10, R11, R12, and R13 are each independently selected from H or optionally substituted C1-10 alkyl;
4. and wherein R1 and R8 may be joined to form a cyclic ring,
or a pharmaceutically acceptable ester, amide, salt, solvate, prodrug, or isomer thereof, with the proviso that when one of R8 and R9 is CH3, then at least one of R10 and R11 is optionally substituted C3-C10 cycloalkyl.

11. The method of claim 8, wherein the therapeutic agent is a compound according to the following structure: 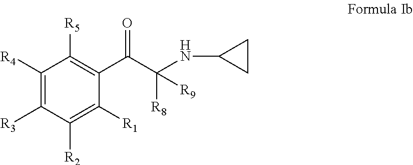
wherein
a. R₁-R₅ are each independently selected from H, OH, optionally substituted C1-4 alkyl, optionally substituted C1-3 alkoxy, optionally substituted C2-4 alkenyl, optionally substituted C2-4 alkynyl, halogen, amino, acylamido, CN, CF₃, NO₂, N₃, CONH₂, CO₂R₁₂, CH₂OR₁₂, NR₁₂R₁₃, NHCOR₁₂, NHCO₂R₁₂, CONR₁₁R₁₃; C1-3 alkylthio, R₁₂SO, R₁₂SO₂, CF₃S, and CF₃SO₂;
b. R₈ and R₉ are each independently selected from H or optionally substituted C1-10 alkyl;
c. R₁₂ and R₁₃ are each independently selected from H or optionally substituted C1-10alkyl; and wherein
d. R₁ and R₈ may be joined to form a cyclic ring,
or a pharmaceutically acceptable ester, amide, salt, solvate, prodrug, or isomer thereof.

12. The method of claim 8, wherein the therapeutic agent is selected from the group consisting Compounds PAL 433, PAL 1122, PAL 1123, PAL 363, PAL 361, PAL 586, PAL 588, PAL 591, PAL 743, PAL 744, PAL 787, PAL 820, PAL 304, PAL 434, PAL 426, PAL 429, and PAL 550, as shown in the table appearing in FIG. 14A.

13. The method of claim 8, wherein the therapeutic agent is a compound according to the structure: 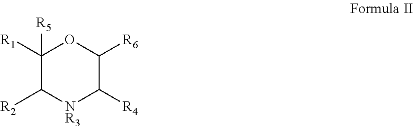
wherein
(a) R₁ is optionally substituted aryl (e.g., naphthyl or phenyl);
(b) R₂ is H or optionally substituted C1-3 alkyl;
(c) R₃ is H, optionally substituted C1-3 alkyl, or benzyl;
(d) R₄ is H or optionally substituted C1-3 alkyl;
(e) R₅ is H or OH; and
(f) R₆ is H or optionally substituted C1-3 alkyl;
with the proviso that when R₂ is CH₃ and R₁ is phenyl, then
(i) the phenyl ring of R₁ is substituted with one or more substituents; or
(ii) R₃ is substituted C1 alkyl or optionally substituted C2-C3 alkyl, or
(iii) one or more of R₄, R₅, and R₆ is not H, or a combination of two or more of (a) through (c);
or a pharmaceutically acceptable ester, amide, salt, solvate, prodrug, or isomer thereof.

14. The method of claim 8, wherein the therapeutic agent is a compound according to the structure: 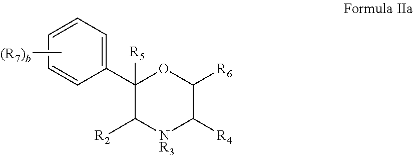
wherein
e. each R7 represents a substituent independently selected from the group consisting of OH, optionally substituted C1-4 alkyl, optionally substituted C1-4 alkoxy, optionally substituted C2-4 alkenyl, optionally substituted C2-4 alkynyl, Cl, F, I, acylamido, CN, CF3, N3, CONH2, CO2R12, CH2OH, CH2OR12, NHCOR12, NHCO2R12, CONR12R13, C1-3 alkylthio, R12SO, R12SO2, CF3S, and CF3SO2,
f. wherein R12 and R13 are each independently selected from H or optionally substituted C1-10 alkyl; and
g. b is an integer from 0-5;
with the proviso that when R2 is CH3, then b is an integer from 1-5 and the phenyl is trans to R2,
or a pharmaceutically acceptable ester, amide, salt, or solvate thereof.

15. The method of claim 8, wherein the therapeutic agent is a compound according to the structure: 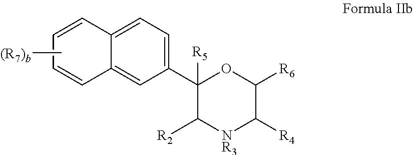
wherein
h. R₂ is H or optionally substituted C1-3 alkyl;
i. R₃ is H, optionally substituted C1-3 alkyl, or benzyl;
j. R₄ is H or optionally substituted C1-3 alkyl;
k. R₅ is H or OH;
l. R₆ is H or optionally substituted C1-3 alkyl;
m. each R₇ represents a substituent independently selected from the group consisting of OH, optionally substituted C1-4 alkyl, optionally substituted C1-3 alkoxy, optionally substituted C2-4 alkenyl, optionally substituted C2-4 alkynyl, halogen, amino, acylamido, CN, CF₃, NO₂, N₃, CONH₂, CO₂R₁₂, CH₂OH, CH₂OR₁₂, NR₁₂R₁₃, NHCOR₁₂, NHCO₂R₁₂, CONR₁₂R₁₃, C1-3 alkylthio, R₁₂SO, R₁₂SO₂, CF₃S, and CF₃SO₂; and
n. c is an integer from 0-7,
or a pharmaceutically acceptable ester, amide, salt, solvate, prodrug, or isomer thereof.

16. The method of claim 8, wherein the therapeutic agent is a compound according to the structure: 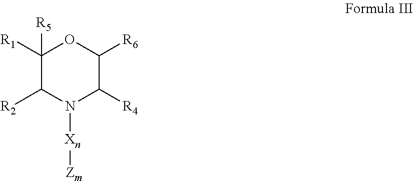
wherein
o. R₁, R₂, R₄, R₅, and R₆ are the same as indicated above for Formula I;
p. X is a chemical moiety, wherein each X may be the same or different;
q. n is an integer from 0 to 50, preferably 1 to 10;
r. Z is a chemical moiety that acts as an adjuvant, wherein each Z may be the same or different, and wherein each Z is different from at least one X; and
s. m is an integer from 0 to 50.

17. The method of claim 8, wherein the therapeutic agent is a compound according to the structure: 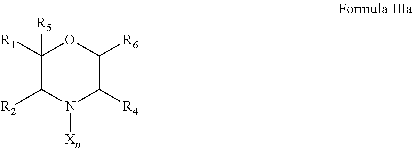
wherein
t. R1, R2, R4, R5, and R6 are the same as indicated above for Formula I;
u. X is a chemical moiety, wherein each X may be the same or different;
v. n is an integer from 0 to 50, preferably 1 to 10;
w. Z is a chemical moiety that acts as an adjuvant, wherein each Z may be the same or different, and wherein each Z is different from at least one X; and
x. m is an integer from 0 to 50.

18. The method of claim 8, wherein the therapeutic agent is a compound according to the structure: 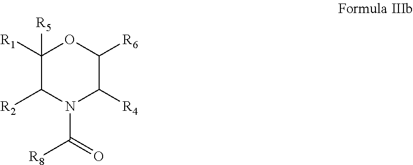
wherein
y. R1, R2, R4, R5, and R6 are the same as indicated above for Formula I;
z. R8 is optionally substituted C1-10 alkyl, optionally substituted C1-10 alkoxy, optionally substituted phenyl, optionally substituted benzyl, or optionally substituted pyridyl,
aa. X is a chemical moiety, wherein each X may be the same or different;
bb. n is an integer from 0 to 50, preferably 1 to 10;
cc. Z is a chemical moiety that acts as an adjuvant, wherein each Z may be the same or different, and wherein each Z is different from at least one X; and
dd. m is an integer from 0 to 50.

19. The method of claim 1, further wherein the therapeutic agent is at least one of:
(a) inactive at the 5-HT2B receptor;
(b) a neutral agonist of the 5-HT2B receptor; and
(c) an inverse agonist of the 5-HT2B receptor 5-HT2B receptor.

20. The method of claim 19, wherein the patient has been diagnosed with an epilepsy syndrome selected from the group consisting of Dravet syndrome, Lennox-Gastaut syndrome, Doose syndrome, West syndrome, and refractory epilepsy.

21. The method of claim 2, wherein an effective dose of the therapeutic agent is administered in a pharmaceutically acceptable carrier.

22. The method of claim 21, wherein the pharmaceutical composition is a formulation adapted to a dosage forms selected from the group consisting of an oral dosage form, an intravenous dosage form, rectal dosage form, subcutaneous dosage form, and a transdermal dosage form.

23. The method of claim 22, wherein the oral dosage form selected from the group consisting of a liquid, a suspension, a tablet, a capsule, a lozenge, and a dissolving strip.
